(19) United States
(12) Patent Application Publication
(10) Pub. No.: US 2012/0071481 A1
(43) Pub. Date: Mar. 22, 2012

(54) ISOXAZOLE-5-CARBOXAMIDE DERIVATIVES

(76) Inventors: Ronald Palin, Banton (GB); Paul David Ratcliffe, Lanarkshire (GB); Steven G. Kultgen, Salt Lake City, UT (US); Koc-Kan Ho, Salt Lake City, UT (US); Andrew Laird Roughton, Port Hope (CA)

(21) Appl. No.: 13/256,553

(22) PCT Filed: Feb. 2, 2010
(86) PCT No.: PCT/US10/22869

§ 371 Date: Dec. 2, 2011

Related U.S. Application Data
(60) Provisional application No. 61/149,722, filed on Feb. 4, 2009.

(30) Foreign Application Priority Data
Feb. 4, 2009 (EP) 09152068.4

(51) Int. Cl. A61K 31/42; C07D 413/12; A61P 25/00; A61K 31/5377; A61K 31/4439; C07D 261/18; A61K 31/454

(52) U.S. Cl. 514/236.8; 548/248; 514/378; 546/209; 514/326; 544/137; 546/272.1; 514/340

(57) ABSTRACT

The present invention relates to isoxazole-3-carboxamide derivative having the general Formula I or a pharmaceutically acceptable salt thereof, to pharmaceutical compositions comprising the same, as well as to the use of said isoxazole-3-carboxamide derivatives for the treatment of TRPV1 mediated disorders, such as acute and chronic pain disorders, acute and chronic neuropathic pain, acute and chronic inflammatory pain, respiratory diseases, and lower urinary tract disorders.

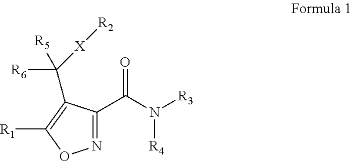

The present invention relates to isoxazole-3-carboxamide derivatives, to pharmaceutical compositions comprising the same and to the use of these isoxazole-3-carboxamide derivatives in the treatment of TRPV1 related disorders.

The vanilloid receptor (VR1 or TRPV1), a non-selective ligand-gated cation channel belonging to the Transient Receptor Channel family (TRP family) of cation channels, is highly expressed on the peripheral termini of small diameter sensory neurones innervating many tissues including skin, bladder, airway and gastrointestinal tract. More specifically TRPV1 receptors are located on a subset Aδ and C fibres, the afferents commonly associated with nociception (Mezey et al., Proc. Natl. Acad. Sci. 97, 3655-3660, 2000). Characterisation of this channel at the molecular level identified it as the target of the vanilloid capsaicin, the main pungent constituent of hot chilli peppers (Caterina et al., Nature 389, 816-824, 1997). Indeed, sensitivity to capsaicin has been used for many years as a marker of nociceptor activity. These, polymodal nociceptors are activated by multiple noxious stimuli including chemical, mechanical and thermal. Study of the functional properties of TRPV1 demonstrated that this receptor shares many properties common to nociceptors including activation by thermal stimuli (>43° C.) and chemicals (including capsaicin and endovanilloids such as N-arachidonoyl-dopamine (NADA) and lipoxygenase metabolites), as well as sensitisation and activation by acidification. Furthermore, inflammatory mediators (including ATP and bradykinin) have been shown to functionally sensitise TRPV1 in vitro. This evidence suggests that TRPV1 has an integral role in the polymodal detection of noxious stimuli and contributes to the transduction of inflammatory pain responses and potentially also peripheral tissue injury (reviewed in Di Marzo et al., Curr. Opin, Neurobiol. 12, 372-379, 2002).

A role for TRPV1 in the detection of painful stimuli is also inferred from data in gene knockout mice. Mice null for TRPV1 show attenuated development of behavioural thermal hyperalgesia after an inflammatory insult (Caterina et al., Science 288, 306-313, 2000, Davis et al., Nature 405, 183-187, 2000). Small diameter sensory neurones from these animals also show altered responses to thermal and acid stimuli. Moreover, altered expression and/or functional activity of TRPV1 has been demonstrated following inflammation and nerve injury in animals models (Arnaya et Brian Res. 963, 190-196, 2003, Rashid et al., Pharm, Exp. Ther. 304, 940-948, 2003, Hong 8, Wiley, J. Bid, Chem, 280, 618.62T 2005) In addition, to a role in pain transduction there is also growing evidence for a role for TRPV1 in regulating afferent and efferent function of sensory nerves and the function of non-neuronal cells. Indeed, altered bladder function, with a higher frequency of low amplitude, non-voiding bladder contractions and an increase in bladder capacity has been observed by in TRPV1 KO mice (Birder et al., Nat. Neurosci. 5, 856-860, 2002). This may involve neuronal TRPV1 and TRPV1 expressed on uroepithelial cells. Thus, there is clear evidence to suggest that agents modulating TRPV1 activity will have utility in not only in pain states and other diseases involving inflammation but also in conditions involving hyperactivity of primary sensory fibres (e.g. bladder overactivity and urge incontinence).

Isoxazole-3-carboxamide derivatives have been disclosed in the International Patent Application WO 2007/067710 (Amphora Discovery Corporation) as modulators of the TRPV1 receptor and useful in the treatment of TRPV1 mediated disorders, such as in the treatment of acute and chronic pain disorders, acute and chronic neuropathic pain, acute and chronic inflammatory pain, respiratory diseases, and lower urinary tract disorders.

There remains a need for additional, more potent, compounds that are useful in the treatment of TRPV1 mediated disorders.

To this end the present invention provides isoxazole-3-carboxamide derivatives having the general Formula I

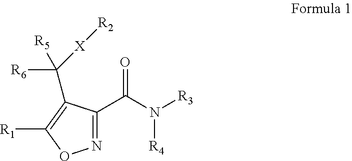

wherein
R₁ is phenyl or pyridyl, optionally substituted by 1-3 substituents selected from halogen, (C₁₋₄)alkyl and (C₁₋₄)alkyloxy, the alkyl and alkyloxy group being optionally substituted with halogen;

### X is O or NR₇;

R₂ is H or (C₁₋₈)alkyl, optionally substituted by OH, (C₃₋₆)cycloalkyl, (C₁₋₃)alkyloxy, (C₁₋₃)alkylSO, (C₁₋₃)alkylSO₂, CN, NR₈R₉, COOR₁₀ or 1 or more halogens; or
R₂ is (C₃₋₁₀)cycloalkyl, (C₃₋₈)cycloalkenyl or (C₃₋₈)cycloalkyl(C₁₋₃)alkyl, each cycloalkyl group optionally substituted by (C₁₋₃)alkyl, hydroxy(C₁₋₃)alkyl, oxo, OH, ═NOH, COOR₁₀ or CN; or
R₂ is phenyl or pyridyl, each of which may be fused to a 5- or 6-membered saturated heterocyclic ring containing 1 or 2 heteroatoms selected from NR₁₁, O and S, and each of which may be substituted by NR₈R₉, halogen, OH, ═NOH, oxo, SH, (C₁₋₃)-alkyl, (C₁₋₃)alkyloxy or hydroxy(C₁₋₃)alkyl, each alkyl group optionally substituted by one or more halogens; or
R₂ together with R₇ and the N to which they are bonded form a saturated 4-8 membered ring, optionally containing a further heteroatom selected from O and S, the ring being optionally substituted by oxo, OH, COOR₁₀ or CON H₂; or R₂ is (CH₂)ₙ-Het;
Het is a saturated 4-8-membered heterocyclic ring containing 1 or 2 heteroatoms selected from NR₁₁, O, S and SO₂, optionally substituted by OH or oxo;
n is 0, 1 or 2;
R₃ is (C₁₋₈)alkyl, (C₂₋₈)alkenyl, or (C₂₋₈)alkynyl, each of which optionally substituted by halogen, OH or phenyl; or
R₃ is (C₃₋₁₀)cycloalkyl, (C₃₋₈)cycloalkenyl or (C₃₋₈)cycloalkyl(C₁₋₃)alkyl, each cycloalkyl group may be fused to a benzo group, and each cycloalkyl group may be substituted by oxo, ═NOH, OH, COOR₁₂, CN, (C₁₋₃)alkyl or hydroxy(C₁₋₃)alkyl; or
R₃ is a saturated 4-8-membered heterocyclic ring containing 1 or 2 heteroatoms selected from N, O, S and SO₂, optionally substituted by hydroxyl or oxo; or
R₃ is phenyl or pyridyl, each of which may be fused to a 5- or 6-membered saturated heterocyclic ring containing 1 or 2 heteroatoms selected from NR₅, O and S, and each of which may be substituted by amino, halogen, hydroxy, hydroxyimino, oxo, mercapto, (C₁₋₃)-alkyl, (C₁₋₃)-alkyloxy or hydroxy(C₁₋₃)alkyl, each alkyl group optionally substituted by one or more halogens; or
R₃ is a bicyclic heteroaromatic ring system containing 1-3 heteroatoms selected from N, O and S, which may be substituted by hydroxy, amino, (C₁₋₃)alkyl or hydroxy-(C₁₋₃)alkyl;
R₄ is H or (C₁₋₄)alkyl; or
R₄ together with R₃ and the N to which they are bonded form a saturated 4-8 membered ring, optionally containing a further heteroatom selected from O, S and SO₂, the ring being optionally substituted by oxo, hydroxyimino, hydroxy, carboxy, carboxamido, (C₁₋₃)alkyl, or hydroxy(C₁₋₃)alkyl or (C₁₋₃)-alkyloxy;
R₅, where present, is H or (C₁₋₄)alkyl;
R₆, where present, is H or (C₁₋₄)alkyl;
R₇ is H, (C₁₋₈)alkyl, (C₃₋₁₀)cycloalkyl, COR₁₃ or SO₂R₁₃;
R₈ and R₉ are independently H or (C₁₋₄)alkyl; or
R₈ and R₉ together with the N to which they are bonded form a 4-7-membered saturated heterocyclic ring;
R₁₀ is H or (C₁₋₄)alkyl;
R₁₁ is H or (C₁₋₄)alkyl;
R₁₂ is H or (C₁₋₄)alkyl;
R₁₃ is (C₁₋₆)alkyl;
or a pharmaceutically acceptable salt thereof.

The term (C₁₋₃)alkyl as used in the definition of Formula I means a branched or un-branched alkyl group having 1-3 carbon atoms, like propyl, ethyl and methyl.

The term hydroxy(C₁₋₃)alkyl means a branched or unbranched alkyl group having 1-3 carbon atoms substituted by 1 or 2 hydroxy groups, such as 3-hydroxypropyl, 2,3-dihydroxypropyl, 2-hydroxyethyl or hydroxymethyl.

The term (C₁₋₄)alkyl as used in the definition of Formula I means a branched or unbranched alkyl group having 1-4 carbon atoms, like butyl, isobutyl, tertiary butyl, propyl, isopropyl, ethyl and methyl.

The term halo(C₁₋₄)alkyl means a branched or unbranched alkyl group having 1-4 carbon atoms substituted by 1-3 halogens. A preferred halo(C₁₋₄)alkyl is CF₃.

In the term (C₁₋₄)alkyloxy, (C₁₋₄)alkyl has the meaning as defined above.

In the term halo(C₁₋₄alkyloxy, halo(C₁₋₄)alkyl has the meaning as defined above.

The term (C₁₋₈)alkyl as used in the definition of Formula I means a branched or unbranched alkyl group having 1-8 carbon atoms, like octyl, hexyl, hexyl, pentyl, isopentyl, butyl, isobutyl, tertiary butyl, propyl, isopropyl, ethyl and methyl.

The term (C₂₋₈)alkenyl means a branched or unbranched alkenyl group having 2-8 carbon atomes, such as ethenyl, propen-2-yl, 2-methyl-propenyla and penten-4-yl.

The term (C₂₋₈)alkynyl means a branched or unbranched alkynyl group having 2-8 carbon atomes, such as ethynyl, propyn-2-yl, pentyn-4-yl and the like.

The term (C₃₋₁₀)cycloalkyl means a cycloalkyl group having 3-10 carbon atoms, like cycloheptyl, cyclohexyl, cyclopentyl, cyclobutyl and cyclopropyl. Also included in this term are bicyclic cycloalkyl groups such as bicyclo[2,2,1]heptan-2-yl, bicyclo[2,2,1]hept-2-enyl, bicyclo[2,2,2]oct-5-enyl, and tricyclic alkyl groups such as adamantyl and the like.

The term (C₃₋₈)cycloalkenyl means a cycloalkenyl group having 3-8 carbon atoms, like cyclooct-3-yl, cyclohex-3-yl and cyclopent-2-yl.

The term a saturated 4-8-membered heterocyclic ring containing a further heteroatom selected from O, S and SO₂, as used in the definition of R₄ together with R₃ and the N to which they are bonded is exemplified by N-morpholinyl, N-thiomorpholinyl and N-thiazolidinyl.

The term a saturated 4-8-membered heterocyclic ring containing a heteroatom selected from O, S and SO₂, as used in the definition of R₃ of formula I is exemplified by tetrahydropyranyl, tetrahydrofuranyl, tetrahydrothiopyranyl, tetrahydrothienyl and N-morpholinyl.

The term halogen means F, Cl, Br or I. Preferred are F and Cl.

There is a preference for isoxazole-3-carboxamide derivative of Formula I wherein R₁ is phenyl, the phenyl being optionally substituted with 1-3 substitutents selected from fluoro, chloro and CF₃.

More preferred are the compounds of Formula I wherein X is NR₇.

One embodiment of the invention relates to the isoxazole-3-carboxamide derivatives of formula I wherein R₂ is (C₁₋₈)alkyl, optionally substituted by OH, (C₃₋₆)cycloalkyl, (C₁₋₃)alkyloxy, (C₁₋₃)alkylSO, (C₁₋₃)alkylSO₂, CN, NR₈R₉, COOR₁₀ or 1 or more halogens; or R₂ is (C₃₋₁₀)cycloalkyl, (C₃₋₈)cycloalkenyl or (C₃₋₈)cycloalkyl(C₁₋₃)alkyl, each cycloalkyl group optionally substituted by (C₁₋₃)alkyl, hydroxy(C₁₋₃)alkyl, oxo, OH, ═NOH, COOR₁₀ or CN.

A further embodiment of the relates to the isoxazole-3-carboxamide derivatives of formula I, wherein R₃ is tetrahydropyranyl or (C₅₋₆)cycloalkyl, substituted by hydroxy or hydroxymethyl.

The isoxazole-3-carboxamide derivatives of the invention may be prepared by methods known in the art of organic chemistry in general.

The compounds of this invention can be prepared from readily available starting materials using the following general procedures. It will be appreciated that where typical or preferred process conditions (i.e reaction temperatures, times, solvents etc.) are given, other process conditions can also be used unless otherwise stated.

Optimum reaction conditions may vary with the particular reactants or solvents used, but such conditions can be determined by one skilled in the art by routine optimisation procedures. The target compounds are synthesised by known reactions outlined in the following schemes. The products are isolated and purified by known standard procedures. Such procedures include (but not limited to) crystallisation, column chromatography or hplc.

The following scheme illustrates the preparation of compounds of Formula D.

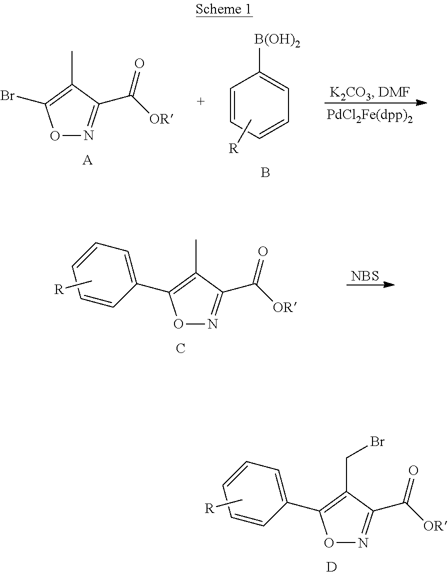

In scheme 1 the compound of Formula C can be prepared by using a Suzuki reaction (Chem. Rev. 95, 2457-2483, 1995) or a modification thereof. Compounds of Formula B can be a boronic acid or a boronic acid ester, and compounds of formula A can be a heteroaromatic halide. Compounds of Formula A and Formula B which serve as starting materials are commercially available or may be prepared by a variety of methods known in the art.

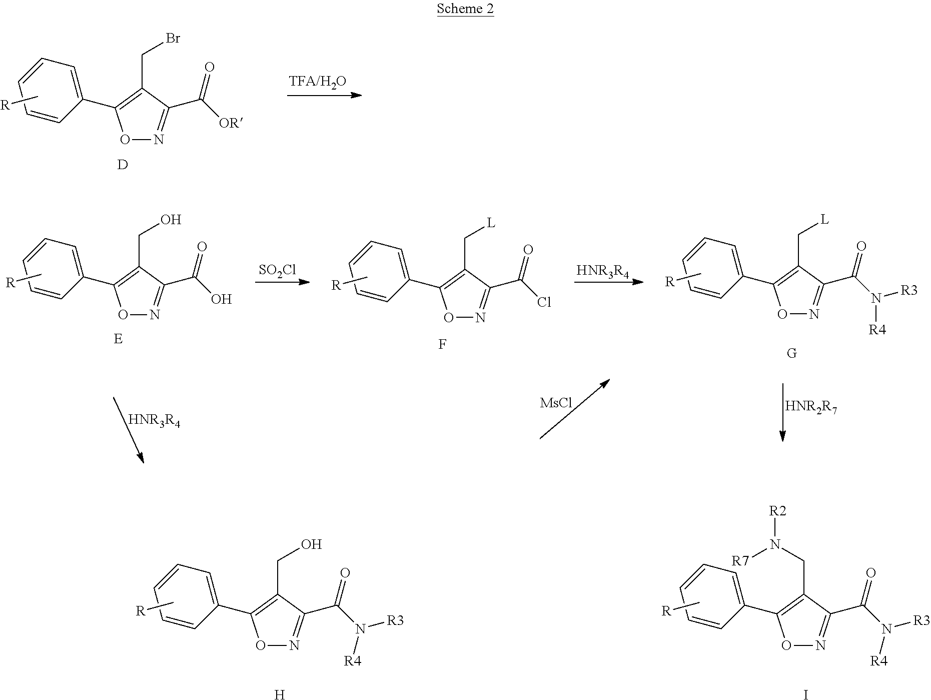

In scheme 2 the compound of formula E can be prepared by hydrolysis of compound of formula D, using a reagent such as trifluoroacetic acid in water. The next step to prepare compound of formula F can be achieved by treating compound of formula E with thionyl chloride to introduce a leaving group, L. Isoxazole-3-carboxamide derivatives of formula G may be prepared from compounds of formula F where L is a halide, by treatment with one or more standard (peptide) coupling reagents well known in the art, such as 0-(7-azabenzotriazol-1-yl)-N,N,N′,N′-tetramethyluronium hexafluorophosphate (HATU), dicyclohexylcarbodiimide (DCC), diisopropylcarbo-diimide (DIC), polyphosphoric acid (PPA) or (benzotriazol-1-yl-oxy-trispyrrolidino-phosphoniumhexafluorophosphate (PYBOP) and further treatment with the appropriate amines NHR₃R₄(J. Am. Chem. Soc., Vol. 108, No. 22, 6950-6960, 1986). Alternatively compounds of formula G can be prepared from compound of formula E by first introducing the appropriate amine NHR₃R₄ with the standard coupling conditions mentioned above to give compounds of formula H. The leaving group, L can then be introduced by converting the hydroxy in compounds of formula H to leaving groups such as meylates using reagents such as methansulphonyl chloride with the appropriate base and solvent. The leaving group, L in compounds of formula G can then be replaced with the appropriate amines NHR₂R₇ to give compounds of formula I. Suitable solvents are aprotic polar solvents such as DMF or acetonitrile although other solvents may be used. Bases such as tertiary amines e.g. triethylamine can be used as well as heteroaromatic bases e.g pyridine. The temperature may be between 0 to 100° C. using either conventional or microwave heating and the reaction time between 1 h and 30 h. The target compounds of formula I can exist in various salt forms such as hydrochloride and trifluoroacetic acid salts.

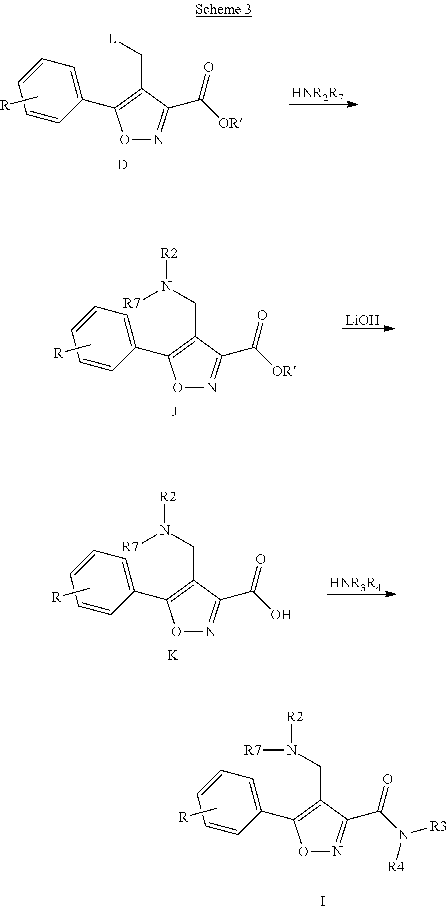

Alternatively compounds of formula I can be prepared by converting compounds of formula D into compounds of formula J (scheme 3) with the appropriate amines NHR₂R₇. Suitable solvents are aprotic polar solvents such as DMF or acetonitrile although other solvents may be used. Bases such as tertiary amines e.g. triethylamine can be used as well as heteroaromatic bases e.g pyridine. The temperature may be between 0 to 100° C. using either conventional or microwave heating and the reaction time between 1 h and 30 h. Compound of formula K can be prepared by hydrolysis of compound of formula J, using a reagent such as Lithium hydroxide, but can include other alkaline earth metal hydroxides, such as sodium hydroxide and potassium hydroxide. Isoxazole-3-carboxamide derivatives of formula I may be prepared from compounds of formula K, by treatment with one or more standard (peptide) coupling reagents well known in the art, such as 0-(7-azabenzotriazol-1-yl)-N,N,N′,N′-tetramethyluronium hexafluorophosphate (HATU), dicyclohexylcarbodiimide (DCC), diisopropylcarbodiimide (DIC), polyphosphoric acid (PPA) or (benzotriazol-1-yl-oxy-trispyrrolidinophosphoniumhexafluorophosphate (PYBOP) and further treatment with the appropriate amines NHR₃R₄ (J. Am. Chem. Soc., Vol. 108, No. 22, 6950-6960, 1986).

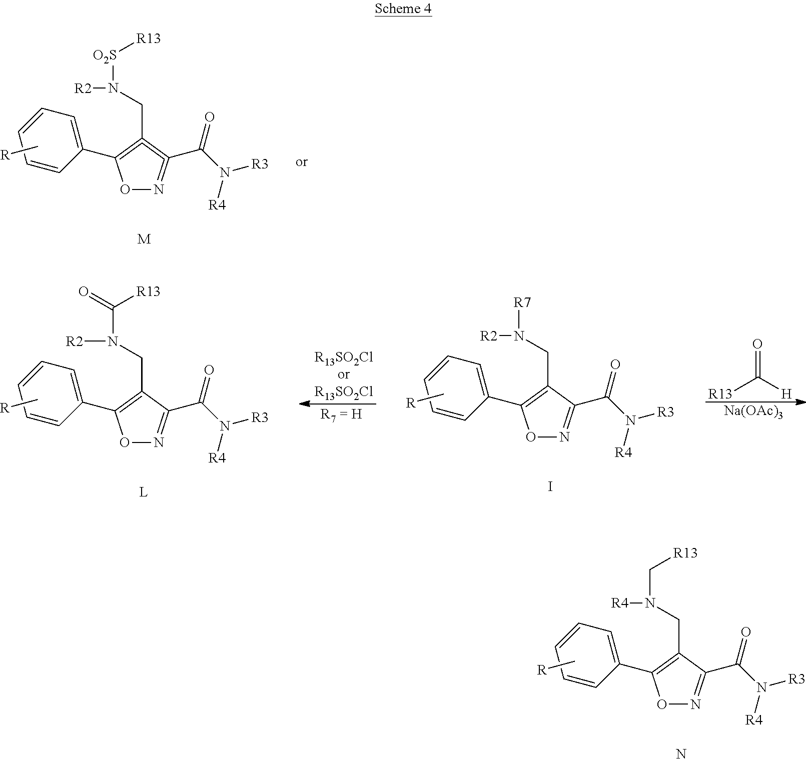

Compounds of formula I can further be derivatised to compounds of formula L and M (scheme 4) by treating with the appropriate acid chloride or sulphoyl chloride respectively in the presence of bases such as tertiary amines e.g. triethylamine as well as heteroaromatic bases e.g pyridine. The temperature may be between 0 to 100° C. using either conventional or microwave heating and the reaction time between 1 h and 30 h. Compounds of formula I can also be derivatised to compounds of formula N using reductive amination conditions such as an appropriate aldehyde or ketone and reducing agent such as sodium borohydride or sodium triacetoxy borohydride.

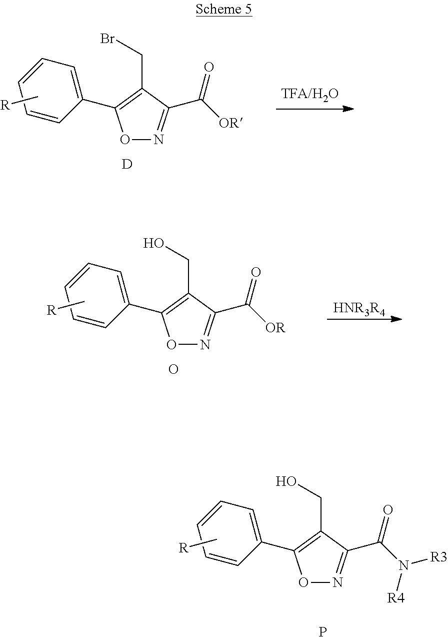

In scheme 5 the compound of formula O can be prepared by hydrolysis of compound of formula D, using a reagent such as trifluoroacetic acid in water. The next step to prepare compound of formula P can be achieved by treating compound of formula 0 with the appropriate amines NHR₃R₄ in the presence of bases such as tertiary amines e.g. triethylamine as well as heteroaromatic bases e.g pyridine. The temperature may be between 0 to 200° C. using either conventional or microwave heating and the reaction time between 1 h and 30 h.

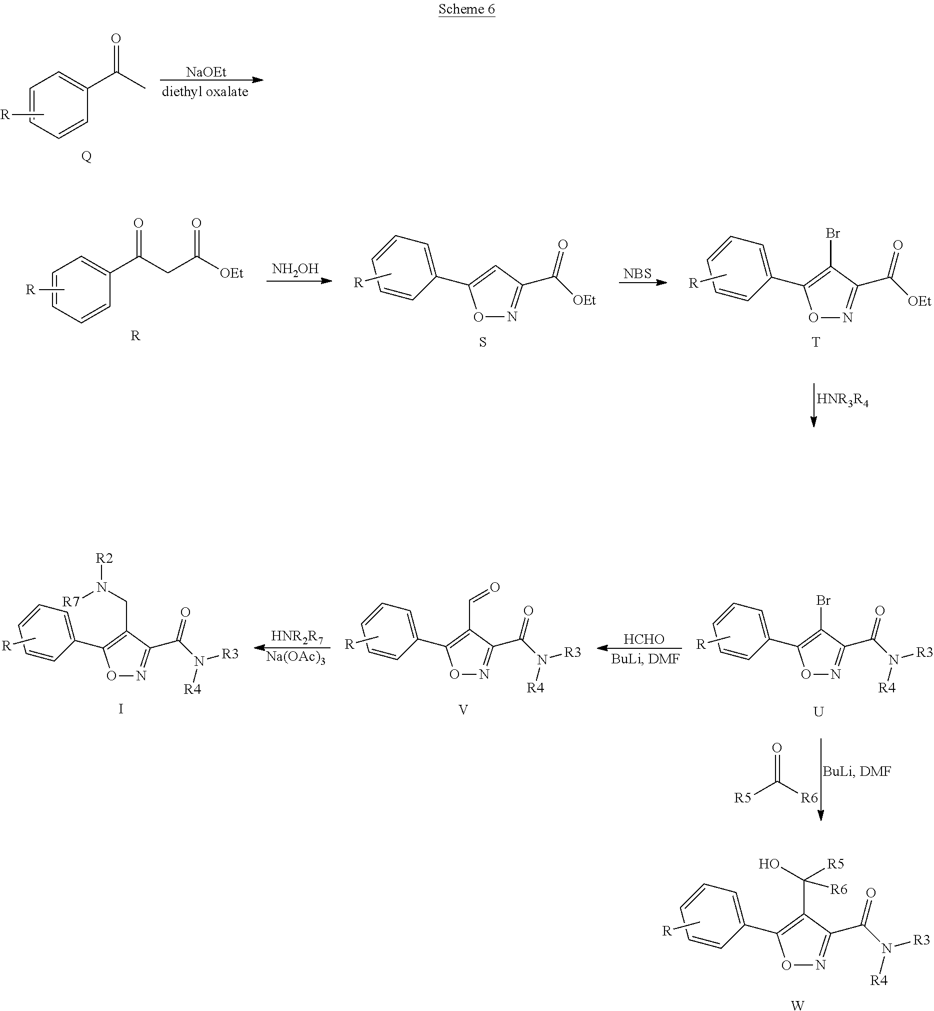

Compounds of formula R may be prepared from compounds of formula Q by condensation with diethyl oxalate in the presence of a suitable base such as sodium ethoxide. Compounds of formula Q which serve as starting materials are commercially available or may be prepared by a variety of methods known in the art. Compounds of formula S, may be prepared from compounds of formula R by treatment with hydroxylamine in a suitable solvent. Compounds of formula T may be prepared from compounds of formula S, using methods well known in the art for halogenating heterocyclic rings. Such as methods described in the general reference Davies, D. T. Aromatic Heterocyclic Chemistry (Oxford University Press: Oxford 1995). The next step to prepare compound of formula U can be achieved by treating compound of formula T with the appropriate amines NHR₃R₄ in the presence of bases such as tertiary amines e.g. triethylamine as well as heteroaromatic bases e.g pyridine. The temperature may be between 0 to 200° C. using either conventional or microwave heating and the reaction time between 1 h and 30 h. Compounds of formula V can be prepared from compounds of formula U using a lithiation procedure and trapping the anion with an aldehyde in the presence of a protic solvent such as DMF. Alternatively trapping the anion with a ketone will result in compounds of formula W. Compounds of formula I can then be derived from compounds of formula V using reductive amination conditions such as an appropriate aldehyde or ketone and reducing agent such as sodium borohydride or sodium triacetoxy borohydride.

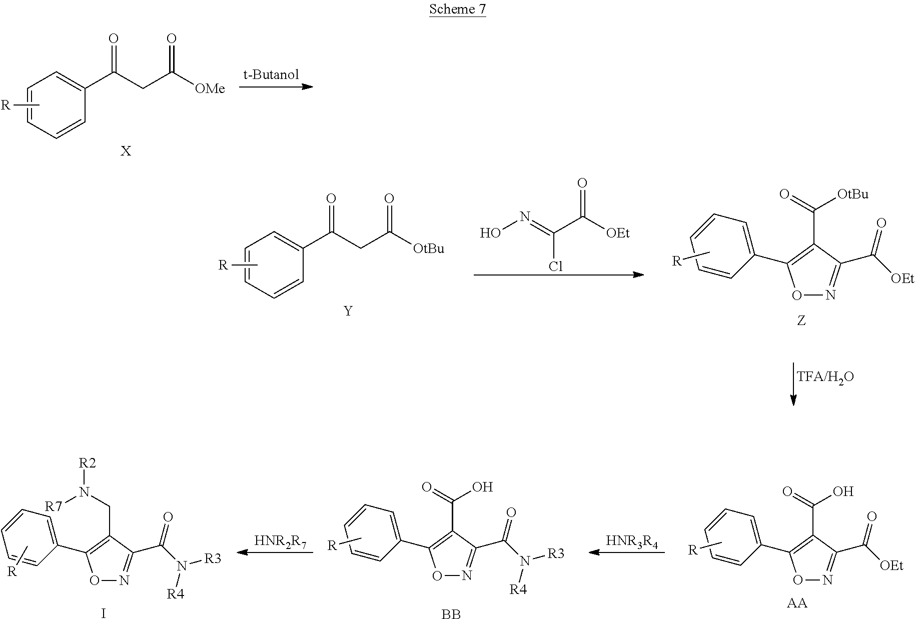

Compounds of formula Y, may be prepared by reaction of compounds of formula X, with t-butanol. Compounds of formula Y can then be condensed with ethyl chlorooximidoacetate in a suitable solvent as described in the general reference Davies, D. T. Aromatic Heterocyclic Chemistry (Oxford University Press: Oxford 1995) to give compounds of formula Z. The compound of formula AA can be prepared by hydrolysis of compound of formula Z, using a reagent such as trifluoroacetic acid in water. The next step to prepare compound of formula BB can be achieved by treating compound of formula AA with the appropriate amines NHR₃R₄ in the presence of bases such as tertiary amines e.g. triethylamine as well as heteroaromatic bases e.g pyridine. The temperature may be between 0 to 200° C. using either conventional or microwave heating and the reaction time between 1 h and 30 h. Isoxazole-3-carboxamide derivatives of formula I may be prepared from compounds of formula BB, by treatment with one or more standard (peptide) coupling reagents well known in the art, such as O-(7-azabenzotriazol-1-yl)-N,N,N′,N′-tetramethyluronium hexafluorophosphate (HATU), dicyclohexylcarbodiimide (DCC), diisopropylcarbodiimide (DIC), polyphosphoric acid (PPA) or (benzotriazol-1-yl-oxy-trispyrrolidinophosphoniumhexafluorophosphate (PYBOP) and further treatment with the appropriate amines NHR₂R₇(J. Am. Chem. Soc., Vol. 108, No. 22, 6950-6960, 1986).

The isoxazole-3-carboxamide derivatives of Formula I and their salts may contain at least one centre of chirality, and exist therefore as stereoisomers, including enantiomers and diastereomers. The present invention includes the aforementioned stereoisomers within its scope and each of the individual R and S enantiomers of the compounds of Formula I and their salts, substantially free, i.e. associated with less than 5%, preferably less than 2%, in particular less than 1% of the other enantiomer, and mixtures of such enantiomers in any proportions including the racemic mixtures containing substantially equal amounts of the two enantiomers.

Methods for asymmetric synthesis or chiral separation whereby the pure stereo-isomers are obtained are well known in the art, e.g. synthesis with chiral induction or starting from commercially available chiral substrates, or separation of stereoisomers, for example using chromatography on chiral media or by crystallisation with a chiral counter-ion.

The present invention also embraces isotopically-labelled isoxazole-3-carboxamide derivatives of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Pharmaceutically acceptable salts may be obtained by treating a free base of a compound of Formula I with a mineral acid such as hydrochloric acid, hydrobromic acid, phosphoric acid and sulfuric acid, or an organic acid such as for example ascorbic acid, citric acid, tartaric acid, lactic acid, maleic acid, malonic acid, fumaric acid, gly-colic acid, succinic acid, propionic acid, acetic acid and methane sulfonic acid.

The compounds of the invention may exist in unsolvated as well as in solvated forms with pharmaceutically acceptable solvents such as water, ethanol and the like. In general, the solvated forms are considered equivalent to the unsolvated forms for the purpose of the invention.

The present invention further provides pharmaceutical compositions comprising an isoxazole-3-carboxamide derivative of the invention, or a pharmaceutically acceptable salt thereof, in admixture with pharmaceutically acceptable auxiliaries, and optionally other therapeutic agents. The term “acceptable” means being compatible with the other ingredients of the composition and not deleterious to the recipients thereof. Compositions include e.g. those suitable for oral, sublingual, subcutaneous, intravenous, epidural, intrathecal, intramuscular, transdermal, pulmonary, local, or rectal administration, and the like, all in unit dosage forms for administration. A preferred route of administration is the oral route.

For oral administration, the active ingredient may be presented as discrete units, such as tablets, capsules, powders, granulates, solutions, suspensions, and the like. For parenteral administration, the pharmaceutical composition of the invention may be presented in unit-dose or multi-dose containers, e.g. injection liquids in predetermined amounts, for example in sealed vials and ampoules, and may also be stored in a freeze dried (lyophilized) condition requiring only the addition of sterile liquid carrier, e.g. water, prior to use.

Mixed with such pharmaceutically acceptable auxiliaries, e.g. as described in the standard reference, Gennaro, A. R. et al., Remington: The Science and Practice of Pharmacy (20th Edition, Lippincott Williams & Wilkins, 2000, see especially Part 5: Pharmaceutical Manufacturing), the active agent may be compressed into solid dosage units, such as pills, tablets, or be processed into capsules, suppositories or patches. By means of pharmaceutically acceptable liquids the active agent may be applied as a fluid composition, e.g. as an injection preparation, in the form of a solution, suspension, emulsion, or as a spray, e.g. a nasal spray.

For making solid dosage units, the use of conventional additives such as fillers, colorants, polymeric binders and the like is contemplated. In general any pharmaceutically acceptable additive which does not interfere with the function of the active compounds may be used. Suitable carriers with which the active agent of the invention may be administered as solid compositions include lactose, starch, cellulose derivatives and the like, or mixtures thereof, used in suitable amounts. For par-enteral administration, aqueous suspensions, isotonic saline solutions and sterile injectable solutions may be used, containing pharmaceutically acceptable dispersing agents and/or wetting agents, such as propylene glycol or butylene glycol.

The invention further includes a pharmaceutical composition, as hereinbefore described, in combination with packaging material suitable for said composition, said packaging material including instructions for the use of the composition for the use as hereinbefore described.

The isoxazole-3-carboxamide derivatives of the invention were found to have modulatory properties at the vanilloid receptor (TRPV1 or VR1) as measured by a fluorescence based calcium flux assay using a Chinese Hamster Ovary cell line in which a human recombinant VR1 receptor had been stably expressed. Methods to construct such recombinant cell lines are well known in the art (Sambrook et al., Molecular Cloning: a Laboratory Manual, Cold Spring Harbor Laboratory Press, Cold Spring Harbor, 2000).

The compounds of the invention are thus useful in the treatment of TRPV1 mediated disorders, such as in the treatment of acute and chronic pain disorders, acute and chronic neuropathic pain, acute and chronic inflammatory pain, respiratory diseases and in lower urinary tract disorders.

The compounds of the invention may be administered to humans in a sufficient amount and for a sufficient amount of time to alleviate the symptoms. Illustratively, dosage levels for humans may be in the range of 0.001-50 mg per kg body weight, preferably in a dosage of 0.01-20 mg per kg body weight.

The invention is illustrated by the following examples:

### General Methods

Flash column chromatography was performed on silica gel. Semi-preparative high pressure liquid chromatography (semi-prep. HPLC) was performed using the method outlined below: X-bridge (C 18.5 μm) 19 mm×50 mm; 10-100% acetonitrile-water over a 8.5 minute gradient followed by 100% acetonitrile for 1.5 minute; 0.1% ammonia buffer; 17 mL/min; detection by UV at 215 nm. Waters Micromass ZQ. ¹H NMR coupling constants are given in Hz.

### EXAMPLE 1

### 4-((Cyclopropylamino)methyl)-N-((1R,3S)-3-hydroxycyclohexyl)-5-(4-(trifluoromethyl)phenyl)isoxazole-3-carboxamide

### A: Ethyl 4-methyl-5-(4-(trifluoromethyl)phenyl)isoxazole-3-carboxylate

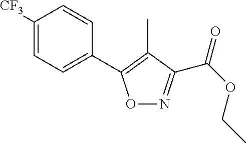

Ethyl 5-bromo-4-methylisoxazole-3-carboxylate (282 mmol, 66 g), 4-(trifluoromethyl)phenylboronic acid (310 mmol, 58.9 g), potassium carbonate (846 mmol, 117 g) and DME (1700 mL) were stirred and purged with nitrogen for 15 minutes. To this was added 1,1-bis(diphenylphosphino)ferrocenedichloropalladium(11) (5.64 mmol, 4.13 g) and the mixture heated to 80° C. for 3 hours. The mixture was filtered through dicalite washing with DME and the filtrate taken to dryness under reduced pressure to leave a dark residue. The residue was passed through a silica gel column eluting with 50% DCM:heptane, then 100% DCM and the product isolated to give the title compound (46.2%). MS (ESI) m/z (M+H⁺): 300.1.

### B: 4-(Bromomethyl)-5-(4-(trifluoromethyl)phenyl)isoxazole-3-carboxylate

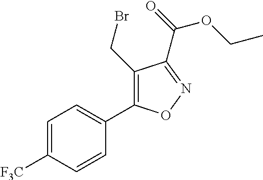

N-Bromosuccinimide (12.03 mmol, 2.14 g) and ethyl 4-methyl-5-(4-(trifluoromethyl)phenyl)isoxazole-3-carboxylate Example 1A (10.03 mmol, 3 g) in carbon terachloride (30 mL) were stirred at reflux whilst irradiating with a UV lamp. After 2 hours the reaction mixture had changed colour to orange. The solvent was removed in vacuo and taken up into dichloromethane. The solid formed (succinimide) was filtered off and the filtrate evaporated to dryness. Purification was carried out by column chromatography eluting with 5% MeOH:DCM. Taken onto the next step (3.29 g, 87%, 2:1 mixture product:starting material).

### C: 4-(Hydroxymethyl)-5-(4-(trifluoromethyl)phenyl)isoxazole-3-carboxylic acid

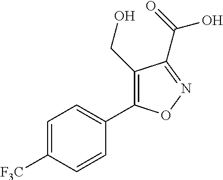

In a 20 mL microwave vial was added ethyl 4-(bromomethyl)-5-(4-(trifluoromethyl)phenyl)isoxazole-3-carboxylate Example 1B (3.97 mmol, 1.5 g) and trifluoroacteic acid in water (25% v/v TFA:water=15 mL) added. The suspension was heated in the microwave at 150° C. for 15 min. before diluting with water. The resultant precipitate was filtered off and dried in an oven at 50° C. under vacuum overnight. The crude product was added to a silica gel column and was eluted with 5% MeOH:DCM. ¹H NMR showed 2:1 mixture of product and hydrolysed starting material from previous step. Taken onto the next step Example 1D (1.19 g, 86%, 2:1 mixture product:starting material).

### D: 4-(Chloromethyl)-5-(4-(trifluoromethyl)phenyl)isoxazole-3-carbonyl chloride

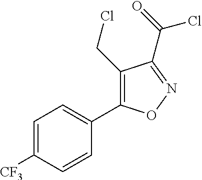

Thionyl chloride (2.86 mmol, 0.21 mL, 0.34 g) and DMF (1.180 mmol, 0.02 mL) were added to a stirred suspension of 4-(hydroxymethyl)-5-(4-(trifluoromethyl)-phenyl)isoxazole-3-carboxylic acid Example 1C (1.18 mmol, 0.34 g) in toluene (2 mL). The reaction mixture was heated to 90° C. for 4 hours before the solvent and excess thionyl chloride were removed under vacuum. Crude material was taken onto the next step.

### E: (1S,3R)-3-(4-(Chloromethyl)-5-(4-(trifluoromethyl)phenyl)isoxazole-3-carboxamido)cyclohexyl acetate

(1S,3R)-3-Aminocyclohexyl acetate (1.42 mmol, 0.22 g) was added as a solution in toluene to a stirred solution of 4-(chloromethyl)-5-(4-(trifluoromethyl)-phenyl)isoxazole-3-carbonyl chloride Example 1D (1.18 mmol, 0.38 g) and triethylamine (1.18 mmol, 0.16 mL, 0.12 g) in toluene. After 6 hours the reaction mixture was partitioned between DCM and water. The organics were collected and concentrated in vacuo, yielding crude product. Taken onto next step.

### F:4-((Cyclopropylamino)methyl)-N-((1R,3S)-3-hydroxycyclohexyl)-5-(4-(trifluoromethyl)phenyl)isoxazole-3-carboxamide

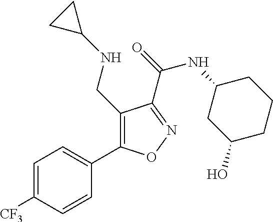

(1S,3R)-3-(4-(Chloromethyl)-5-(4-(trifluoromethyl)phenyl)isoxazole-3-carboxamido)cyclohexyl acetate Example 1E (0.23 mmol, 100 mg), cyclopropylamine (2.25 mmol, 128 mg), DIPEA (0.90 mmol, 116 mg) and acetonitrile (1 mL) were combined in a vial and treated with microwaves at 165° C. for 15 mins. LiOH (1M, 0.5 mL) was added and the reaction stirred overnight. The reaction mixture was partitioned between DCM and water. The organics were concentrated in vacuo and the residue purified by preparative LCMS yielding the title compound (10 mg, 11%). MS (ESI) m/z (M+H⁺): 424.5.

The method of Example 1 was further used to prepare the following compounds using alternative amines in step Example 1E instead of (1S,3R)-3-aminocyclohexyl acetate.

### EXAMPLE 2

### 2A: N-((1R,3S)-3-Hydroxycyclohexyl)-5-(4-(trifluoromethyl)phenyl)-4-(((S)-1,1,1-trifluoropropan-2-ylamino)methyl)isoxazole-3-carboxamide

MS (ESI) m/z (M+H⁺): 480.5.

### 2B: 4-((Cyclobutylmethylamino)methyl)-N-((1R,3S)-3-hydroxycyclohexyl)-5-(4-(trifluoromethyl)phenyl)isoxazole-3-carboxamide

MS (ESI) m/z (M+H⁺): 452.5.

### 2C: N-((1R,3S)-3-Hydroxycyclohexyl)-4-((isobutylamino)methyl)-5-(4-(trifluoromethyl)phenyl)isoxazole-3-carboxamide

MS (ESI) m/z (M+H⁺): 440.5.

### 2D: 4-(((R)-1-Hydroxy-3-methylbutan-2-ylamino)methyl)-N-((1R,3S)-3-hydroxycyclohexyl)-5-(4-(trifluoromethyl)phenyl)isoxazole-3-carboxamide

MS (ESI) m/z (M+H⁺): 470.5.

### 2E: N-((1R,3S)-3-Hydroxycyclohexyl)-4-((S)-tetrahydrofuran-2-yl)methylamino)-methyl)-5-(4-(trifluoromethyl)phenyl)isoxazole-3-carboxamide

MS (ESI) m/z (M+H⁺): 468.5.

### 2F: 4-((3-Hydroxy-2,2-dimethylpropylamino)methyl)-N-((1R,3S)-3-hydroxycyclohexyl)-5-(4-(trifluoromethyl)phenyl)isoxazole-3-carboxamide

MS (ESI) m/z (M+H⁺): 470.5.

### 2G: 4-((Cyclopropylmethylamino)methyl)-N-((1R,3S)-3-hydroxycyclohexyl)-5-(4-(trifluoromethyl)phenyl)isoxazole-3-carboxamide

MS (ESI) m/z (M+H⁺): 438.5.

### 2H: N-((1R,3S)-3-Hydroxycyclohexyl)-4-((3,3,3-trifluoro-2-hydroxypropylamino)methyl)-5-(4-(trifluoromethyl)phenyl)isoxazole-3-carboxamide

MS (ESI) m/z (M+H⁺): 496.5.

### 2I: N-((1R,3S)-3-Hydroxycyclohexyl)-4-(((2-methoxyethyl)(methyl)amino)methyl)-5-(4-(trifluoromethyl)phenyl)isoxazole-3-carboxamide

MS (ESI) m/z (M+H⁺): 456.5.

### 2J: 4-((Ethyl(isopropyl)amino)methyl)-N-((1R,3S)-3-hydroxycyclohexyl)-5-(4-(trifluoromethyl)phenyl)isoxazole-3-carboxamide

MS (ESI) m/z (M+H⁺): 454.5.

### 2K: 4-(((S)-1-Hydroxy-3-methylbutan-2-ylamino)methyl)-N-((1R,3S)-3-hydroxycyclohexyl)-5-(4-(trifluoromethyl)phenyl)isoxazole-3-carboxamide

MS (ESI) m/z (M+H⁺): 470.5.

### 2L: N-((1R,3S)-3-Hydroxycyclohexyl)-4-(((R)-tetrahydrofuran-3-ylamino)methyl)-5-(4-(trifluoromethyl)phenyl)isoxazole-3-carboxamide

MS (ESI) m/z (M+H⁺): 454.5.

### 2M: 4-((Cyclobutylamino)methyl)-N-((1R,3S)-3-hydroxycyclohexyl)-5-(4-(trifluoromethyl)phenyl)isoxazole-3-carboxamide

MS (ESI) m/z (M+H⁺): 438.5.

### 2N: N-((1R,3S)-3-Hydroxycyclohexyl)-4-((isopropyl(methyl)amino)methyl)-5-(4-(trifluoromethyl)phenyl)isoxazole-3-carboxamide

MS (ESI) m/z (M+H⁺): 440.5.

### 2O: N-((1R,3S)-3-Hydroxycyclohexyl)-4-((tetrahydro-2H-pyran-4-ylamino)methyl)-5-(4-(trifluoromethyl)phenyl)isoxazole-3-carboxamide

MS (ESI) m/z (M+H⁺): 468.5.

### 2P: N-((1R,3S)-3-Hydroxycyclohexyl)-4-((methyl(1-methylpiperidin-4-yl)amino)methyl)-5-(4-(trifluoromethyl)phenyl)isoxazole-3-carboxamide

MS (ESI) m/z (M+H⁺): 495.5.

### 2Q: N-((1R,3S)-3-Hydroxycyclohexyl)-4-((methyl(2-(methylsulfonyl)ethyl)amino)-methyl)-5-(4-(trifluoromethyl)phenyl)isoxazole-3-carboxamide

MS (ESI) m/z (M+H⁺): 504.5.

### 2R: 4-((4-Carbamoylpiperidin-1-yl)methyl)-N-((1R,3S)-3-hydroxycyclohexyl)-5-(4-(trifluoromethyl)phenyl)isoxazole-3-carboxamide

MS (ESI) m/z (M+H⁺): 495.5.

### 2S: 4-((Cyclopropylamino)methyl)-N-((1R,3S)-3-hydroxycyclohexyl)-5-(4-(trifluoro-methyl)phenyl)isoxazole-3-carboxamide

MS (ESI) m/z (M+H⁺): 424.5.

### 2T: N-((1R,3S)-3-Hydroxycyclohexyl)-4-((isopropylamino)methyl)-5-(4-(trifluoro-methyl)phenyl)isoxazole-3-carboxamide

MS (ESI) m/z (M+H⁺): 426.5.

### 2U: N-((1R,3S)-3-Hydroxycyclohexyl)-4-((2-methoxyethylamino)methyl)-5-(4-(trifluoromethyl)phenyl)isoxazole-3-carboxamide

MS (ESI) m/z (M+H⁺): 442.5.

### EXAMPLE 3

### N-Cyclopentyl-4-(methylamino)methyl)-5-(4-(trifluoromethyl)phenyl)isoxazole-3-carboxamide

### A: N-Cyclopentyl-4-(hydroxymethyl)-5-(4-(trifluoromethyl)phenyl)isoxazole-3-carboxamide

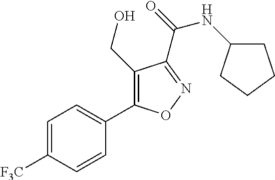

Cyclophos/PPA 50% in EtOAc (7.83 mmol, 4.66 mL, 4.99 g) was added to a cooled (ice/water bath) solution of cyclopentylamine (5.22 mmol, 0.516 mL, 0.445 g), 4-(hydroxymethyl)-5-(4-(trifluoromethyl)phenyl)isoxazole-3-carboxylic acid Example 1C (5.22 mmol, 1.5 g) and triethylamine (15.67 mmol, 2.20 mL, 1.59 g) in DCM (80 mL). The reaction mixture was stirred at room temperature for 2 hours. The reaction was then quenched by the addition of sodium carbonate and the resulting solution stirred for 15 mins. The reaction mixture was diluted with DCM and filtered through a hydrophobic frit, the DCM fraction was then concentrated under reduced pressure. The crude product was added to a silica gel column and was eluted with 5% MeOH:DCM to yield the title compound. MS (ESI) m/z (M+H⁺): 355.5.

### B: (3-(Cyclopentylcarbamoyl)-5-(4-(trifluoromethyl)phenyl)isoxazol-4-yl)methyl methanesulfonate

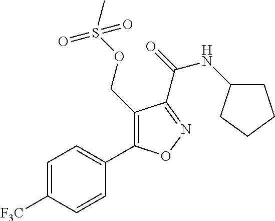

N-Cyclopentyl-4-(hydroxymethyl)-5-(4-(trifluoromethyl)phenyl)isoxazole-3-carboxamide (3A: 0.64 mmol, 0.23 g) in dichloromethane was cooled in an ice bath and methansulphonyl chloride (0.95 mmol, 0.07 mL 0.11 g) and triethylamine (1.27 mmol, 0.18 mL, 0.13 g) were added. The reaction was allowed to warm to room temperature for 4 hours. The reaction mixture was washed with sodium bicarbonate and the organics dried through a hydrophobic frit and concentrated under vacuum. Taken onto next stage as mixture of mesylate and chloro-compounds (0.27 g, 100%). MS (ESI) m/z (M+H⁺): 433.5.

### C: N-Cyclopentyl-4-(methylamino)methyl)-5-(4-(trifluoromethyl)phenyl)isoxazole-3-carboxamide

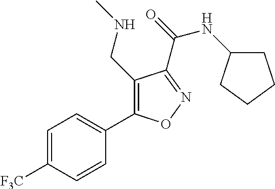

A solution of (3-(cyclopentylcarbamoyl)-5-(4-(trifluoromethyl)phenyl)isoxazol-4-yl)methyl methanesulfonate Example 3B (0.12 mmol, 50 mg), DIPEA (0.46 mmol, 0.08 mL, 59.8 mg) and methylamine (1.16 mmol, 0.10 mL, 90 mg) (40% water) in acetonitrile (1.0 mL) was heated in the microwave at 165° C. for 900 sec. Reaction mixture was worked up by partitioning the mixture between DCM and water; DCM was washed with water twice followed by brine and the collected organics dried over MgSO₄. The crude product was added to a SCX column and was eluted with dichloromethane, methanol, then 2M NH₃ in MeOH. The basic fractions were concentrated and evaporated to dryness to give the title compound (24.4 mg, 57%). MS (ESI) m/z (M+H⁺): 368.5.

The method of Example 3 was further used to prepare the following compounds using alternative amines instead of methylamine.

### EXAMPLE 4

### 4A: N-Cyclopentyl-5-(4-(trifluoromethyl)phenyl)-4-((1,1,1-trifluoropropan-2-ylamino)methyl)isoxazole-3-carboxamide

MS (ESI) m/z (M+H⁺): 450.5.

### 4B: N-Cyclopentyl-4-((Pyridin-2-ylamino)methyl)-5-(4-(trifluoromethyl)phenyl)-isoxazole-3-carboxamide

MS (ESI) m/z (M+H⁺): 431.5.

### 4C: N-Cyclopentyl-4-((4-fluorobenzylamino)methyl)-5-(4-(trifluoromethyl)phenyl)isoxazole-3-carboxamide

MS (ESI) m/z (M+H⁺): 462.5.

### 4D: N-Cyclopentyl-4-((1-methyl-1H-pyrazol-3-ylamino)methyl)-5-(4-(trifluoromethyl)phenyl)isoxazole-3-carboxamide

MS (ESI) m/z (M+H⁺): 434.5.

### 4E: N-Cyclopentyl-4-((isobutylamino)methyl)-5-(4-(trifluoromethyl)phenyl)isoxazole-3-carboxamide

MS (ESI) m/z (M+H⁺): 410.5.

### 4F: N-cyclopentyl-4-(((tetrahydro-2H-pyran-4-yl)methylamino)methyl)-5-(4-(trifluoromethyl)phenyl)isoxazole-3-carboxamide

MS (ESI) m/z (M+H⁺): 452.5.

### 4G: 4-((Cyclobutylmethylamino)methyl)-N-cyclopentyl-5-(4-(trifluoromethyl)phenyl)isoxazole-3-carboxamide

MS (ESI) m/z (M+H⁺): 422.5.

### 4H: N-Cyclopentyl-4-((tetrahydro-2H-pyran-4-ylamino)methyl)-5-(4-(trifluoromethyl)phenyl)isoxazole-3-carboxamide

MS (ESI) m/z (M+H⁺): 438.5.

### 4I: 4-((Benzylamino)methyl)-N-cyclopentyl-5-(4-(trifluoromethyl)phenyl)isoxazole-3-carboxamide

MS (ESI) m/z (M+H⁺): 444.5.

### 4J: N-Cyclopentyl-4-(((2-methoxyethyl)(methyl)amino)methyl)-5-(4-(trifluoromethyl)phenyl)isoxazole-3-carboxamide

MS (ESI) m/z (M+H⁺): 426.5.

### 4K: N-Cyclopentyl-4-((cyclopropylmethylamino)methyl)-5-(4-(trifluoromethyl)phenyl)isoxazole-3-carboxamide

MS (ESI) m/z (M+H⁺): 408.5.

### 4L: N-Cyclopentyl-4-((2-methyl-1-morpholinopropan-2-ylamino)methyl)-5-(4-(trifluoromethyl)phenyl)isoxazole-3-carboxamide 2,2,2-trifluoroacetate

MS (ESI) m/z (M+H⁺): 495.5.

### 4M: N-Cyclopentyl-4-((1-(hydroxymethyl)cyclopentylamino)methyl)-5-(4-(trifluoromethyl)phenyl)isoxazole-3-carboxamide

MS (ESI) m/z (M+H⁺): 452.5.

### 4N: N-Cyclopentyl-4-((3-hydroxy-3-methylbutylamino)methyl)-5-(4-(trifluoromethyl)phenyl)isoxazole-3-carboxamide

MS (ESI) m/z (M+H⁺): 440.5.

### 4O: N-Cyclopentyl-4-((2-(piperidin-1-yl)ethylamino)methyl)-5-(4-(trifluoromethyl)phenyl)isoxazole-3-carboxamide

MS (ESI) m/z (M+H⁺): 465.5.

### 4P: N-Cyclopentyl-4-(((2-hydroxyethyl)(isopropyl)amino)methyl)-5-(4-(trifluoromethyl)phenyl)isoxazole-3-carboxamide

MS (ESI) m/z (M+H⁺): 440.5.

### 4Q: N-Cyclopentyl-4-((cyclopropylamino)methyl)-5-(4-(trifluoromethyl)phenyl)-isoxazole-3-carboxamide

MS (ESI) m/z (M+H⁺): 394.5.

### 4R: Methyl-2-((3-(cyclopentylcarbamoyl)-5-(4-(trifluoromethyl)phenyl)isoxazol-4-yl)methylamino)propanoate

MS (ESI) m/z (M+H⁺): 440.5.

### 4S: N-Cyclopentyl-4-(morpholinomethyl)-5-(4-(trifluoromethyl)phenyl)isoxazole-3-carboxamide

MS (ESI) m/z (M+H⁺): 424.5.

### 4T: N-Cyclopentyl-4-((dimethylamino)methyl)-5-(4-(trifluoromethyl)phenyl)isoxazole-3-carboxamide

MS (ESI) m/z (M+H⁺): 382.5.

### 4U: N-Cyclopentyl-4-((2-(methylsulfonyl)ethylamino)methyl)-5-(4-(trifluoromethyl)phenyl)isoxazole-3-carboxamide

MS (ESI) m/z (M+H⁺): 460.5.

### EXAMPLE 5

### (S)—N-(3-Methylbutan-2-yl)-4-((4-(trifluoromethyl)benzylamino)methyl)-5-(4-(trifluoromethyl)phenyl)isoxazole-3-carboxamide

### A: (S)-4-(Hydroxymethyl)-N-(3-methylbutan-2-yl)-5-(4-(trifluoromethyl)phenyl)-isoxazole-3-carboxamide

Cyclophos/PPA 50% in EtOAc (7.83 mmol, 4.66 mL, 4.99 g) was added to a cooled (ice/water bath) solution of 4-(hydroxymethyl)-5-(4-(trifluoromethyl)phenyl)-isoxazole-3-carboxylic acid Example 1D (5.22 mmol, 1.5 g) and triethylamine (15.67 mmol, 2.20 mL, 1.59 g) in DCM (80 mL). The reaction mixture was stirred at room temperature for 1 hour. The reaction was quenched by the addition of sodium carbonate and the resulting solution stirred for 15 mins. The reaction mixture was diluted with DCM and filtered through a hydrophobic frit, the DCM fraction was then concentrated. The crude product was added to a silica gel column and was eluted with 5% MeOH:DCM to yield the title compound. MS (ESI) m/z (M+H⁺): 357.5.

### B: (S)-(3-(3-Methylbutan-2-ylcarbamoyl)-5-(4-(trifluoromethyl)phenyl)isoxazol-4-yl)-methyl methanesulfonate

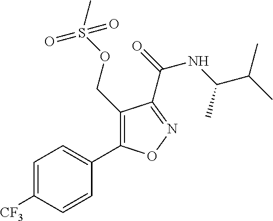

(S)-4-(Hydroxymethyl)-N-(3-methylbutan-2-yl)-5-(4-(trifluoromethyl)phenyl)-isoxazole-3-carboxamide Example 5A (2.60 mmol, 0.93 g) in DCM was cooled in an ice bath and methanesulphonyl chloride (3.90 mmol, 0.30 mL, 0.45 g), and triethylamine (5.20 mmol, 0.73 mL, 0.53 g) were added. The reaction was stirred for 1.5 hours. The reaction mixture was washed with sodium bicarbonate and the organics dried through a hydrophobic frit and concentrated under reduced pressure to yield the title compound (1.12 g). MS (ESI) m/z (M+H⁺): 435.5.

### C: (S)—N-(3-Methylbutan-2-yl)-4-((4-(trifluoromethyl)benzylamino)methyl)-5-(4-(trifluoromethyl)phenyl)isoxazole-3-carboxamide

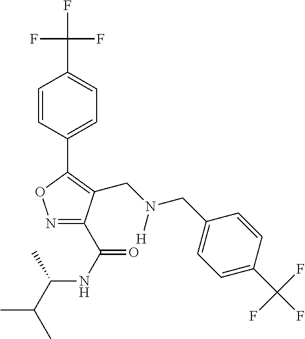

A solution of (S)-(3-(3-methylbutan-2-ylcarbamoyl)-5-(4-(trifluoromethyl)-phenyl)isoxazol-4-yl)methyl methanesulfonate Example 5B (0.07 mmol, 30 mg), DIPEA (0.27 mmol, 0.05 mL, 35.7 mg) and 4-(trifluoromethyl)benzylamine (0.69 mmol, 120 mg) in acetonitrile (1 mL) was heated in the microwave at 165° C. for 900 s. The reaction mixture was diluted with DCM and washed twice with water before pouring through a hydrophobic frit. The crude product was added to a SCX column and was eluted with DCM, MeOH and then 2M NH₃ in MeOH. The crude product was added to a preparative LCMS (basic) column and was eluted with acetonitrile:water. Fractions containing product were evaporated to dryness using a Genevac evaporating system to yield the title compound. MS (ESI) m/z (M+H⁺): 514.5.

The method of Example 5 was further used to prepare the following compounds using alternative amines instead of 4-(trifluoromethyl)benzylamine.

### EXAMPLE 6

### 6A: (S)—N-(3-Methylbutan-2-yl)-4-((pyridin-2-ylamino)methyl)-5-(4-(trifluoromethyl)phenyl)isoxazole-3-carboxamide

MS (ESI) m/z (M+H⁺): 433.5.

### 6B: (S)-4-((Cyclopropylamino)methyl)-N-(3-methylbutan-2-yl)-5-(4-(trifluoromethyl)phenyl)isoxazole-3-carboxamide

MS (ESI) m/z (M+H⁺): 396.5.

### 6C: N—((S)-3-Methylbutan-2-yl)-5-(4-(trifluoromethyl)phenyl)-4-(((R)-1,1,1-trifluoropropan-2-ylamino)methyl)isoxazole-3-carboxamide

MS (ESI) m/z (M+H⁺): 452.5.

### 6D: (S)-4-((3-Fluorobenzylamino)methyl)-N-(3-methylbutan-2-yl)-5-(4-(trifluoromethyl)phenyl)isoxazole-3-carboxamide

MS (ESI) m/z (M+H⁺): 464.5.

### 6E: Ethyl-3-((3-((S)-3-methylbutan-2-ylcarbamoyl)-5-(4-(trifluoromethyl)phenyl)isoxazol-4-yl)methylamino)butanoate

MS (ESI) m/z (M+H⁺): 470.5.

### 6F: (S)-4-((Diethylamino)methyl)-N-(3-methylbutan-2-yl)-5-(4-(trifluoromethyl)phenyl)isoxazole-3-carboxamide

MS (ESI) m/z (M+H⁺): 412.5.

### 6G: 4-(((R)-1-Hydroxy-3-methylbutan-2-ylamino)methyl)-N—((S)-3-methylbutan-2-yl)-5-(4-(trifluoromethyl)phenyl)isoxazole-3-carboxamide

MS (ESI) m/z (M+H⁺): 442.5.

### 6H: (S)-4-((4-Fluorophenylamino)methyl)-N-(3-methylbutan-2-yl)-5-(4-(trifluoromethyl)phenyl)isoxazole-3-carboxamide

MS (ESI) m/z (M+H⁺): 450.5.

### 6I: (S)-4-((Cyclopropylmethylamino)methyl)-N-(3-methylbutan-2-yl)-5-(4-(trifluoromethyl)phenyl)isoxazole-3-carboxamide

MS (ESI) m/z (M+H⁺): 410.5.

### 6J: (S)-4-((2-Cyclopropylethylamino)methyl)-N-(3-methylbutan-2-yl)-5-(4-(trifluoromethyl)phenyl)isoxazole-3-carboxamide

MS (ESI) m/z (M+H⁺): 424.5.

### 6K: (S)-4-((Benzylamino)methyl)-N-(3-methylbutan-2-yl)-5-(4-(trifluoromethyl)phenyl)isoxazole-3-carboxamide

MS (ESI) m/z (M+H⁺): 446.5.

### 6L: (S)-4-((Ethyl(isopropyl)amino)methyl)-N-(3-methylbutan-2-yl)-5-(4-(trifluoromethyl)phenyl)isoxazole-3-carboxamide

MS (ESI) m/z (M+H⁺): 426.5.

### 6M: (S)—N-(3-Methylbutan-2-yl)-4-((phenylamino)methyl)-5-(4-(trifluoromethyl)phenyl)isoxazole-3-carboxamide

MS (ESI) m/z (M+H⁺): 432.5.

### 6N: N—((S)-3-Methylbutan-2-yl)-4-((3,3,3-trifluoro-2-hydroxypropylamino)methyl)-5-(4-(trifluoromethyl)phenyl)isoxazole-3-carboxamide

MS (ESI) m/z (M+H⁺): 468.5.

### 6O: (S)—N-(3-Methylbutan-2-yl)-5-(4-(trifluoromethyl)phenyl)-4-((5-(trifluoromethyl)pyridin-2-ylamino)methyl)isoxazole-3-carboxamide

MS (ESI) m/z (M+H⁺): 501.5.

### 6P: (S)-4-((((1,5-Dimethyl-1H-pyrazol-3-yl)methyl)(methyl)amino)methyl)-N-(3-methylbutan-2-yl)-5-(4-(trifluoromethyl)phenyl)isoxazole-3-carboxamide

MS (ESI) m/z (M+H⁺): 478.5.

### 6Q: (S)-4-((Isopropyl(methyl)amino)methyl)-N-(3-methylbutan-2-yl)-5-(4-(trifluoromethyl)phenyl)isoxazole-3-carboxamide

MS (ESI) m/z (M+H⁺): 412.5.

### 6R: (S)-4-((Cyclobutylamino)methyl)-N-(3-methylbutan-2-yl)-5-(4-(trifluoromethyl)phenyl)isoxazole-3-carboxamide

MS (ESI) m/z (M+H⁺): 410.5.

### 6S: (S)-4-((Cyanomethylamino)methyl)-N-(3-methylbutan-2-yl)-5-(4-(trifluoromethyl)phenyl)isoxazole-3-carboxamide

MS (ESI) m/z (M+H⁺): 395.5.

### 6T: 4-(((S)-1-Hydroxy-3-methylbutan-2-ylamino)methyl)-N—((S)-3-methylbutan-2-yl)-5-(4-(trifluoromethyl)phenyl)isoxazole-3-carboxamide

MS (ESI) m/z (M+H⁺): 442.5.

### 6U: (S)-4-((3-Hydroxy-2,2-dimethylpropylamino)methyl)-N-(3-methylbutan-2-yl)-5-(4-(trifluoromethyl)phenyl)isoxazole-3-carboxamide

MS (ESI) m/z (M+H⁺): 442.5.

### 6V: (S)—N-(3-Methylbutan-2-yl)-4-((tetrahydro-2H-pyran-4-ylamino)methyl)-5-(4-(trifluoromethyl)phenyl)isoxazole-3-carboxamide

MS (ESI) m/z (M+H⁺): 440.5.

### 6W: (S)-4-((Dimethylamino)methyl)-N-(3-methylbutan-2-yl)-5-(4-(trifluoromethyl)phenyl)isoxazole-3-carboxamide

MS (ESI) m/z (M+H⁺): 384.5.

### 6X: (S)-4-((1-(Dimethylamino)-2-methylpropan-2-ylamino)methyl)-N-(3-methylbutan-2-yl)-5-(4-(trifluoromethyl)phenyl)isoxazole-3-carboxamide

MS (ESI) m/z (M+H⁺): 455.5.

### 6Y: (S)-4-((2-Hydroxy-2-methylpropylamino)methyl)-N-(3-methylbutan-2-yl)-5-(4-(trifluoromethyl)phenyl)isoxazole-3-carboxamide

MS (ESI) m/z (M+H⁺): 428.5.

### 6Z: (S)-4-(((1-Hydroxycyclopropyl)methylamino)methyl)-N-(3-methylbutan-2-yl)-5-(4-(trifluoromethyl)phenyl)isoxazole-3-carboxamide

MS (ESI) m/z (M+H⁺): 426.5.

### 6AA: (S)-4-((Methylamino)methyl)-N-(3-methylbutan-2-yl)-5-(4-(trifluoromethyl)phenyl)isoxazole-3-carboxamide

MS (ESI) m/z (M+H⁺): 370.5.

### 6AB: (S)-4-((2-Iminopyridin-1(2H)-yl)methyl)-N-(3-methylbutan-2-yl)-5-(4-(trifluoromethyl)phenyl)isoxazole-3-carboxamide

MS (ESI) m/z (M+H⁺): 433.5.

### 6AC: (S)-4-((2-Imino-4-(trifluoromethyl)pyridin-1(2H)-ylamino)methyl)-N-(3-methylbutan-2-yl)-5-(4-(trifluoromethyl)phenyl)isoxazole-3-carboxamide

MS (ESI) m/z (M+H⁺): 501.5.

### 6AD: 4-((3-Hydroxycyclohexylamino)methyl)-N—((S)-3-methylbutan-2-yl)-5-(4-(trifluoromethyl)phenyl)isoxazole-3-carboxamide

MS (ESI) m/z (M+H⁺): 454.5.

### EXAMPLE 7

### N-((1s,4s)-4-Hydroxycyclohexyl)-4-((isobutylamino)methyl)-5-(4-(trifluoromethyl)phenyl)isoxazole-3-carboxamide

### A: 4-(Chloromethyl)-5-(4-(trifluoromethyl)phenyl)-N-((1s,4s)-4-(trimethylsilyloxy)-cyclohexyl)isoxazole-3-carboxamide

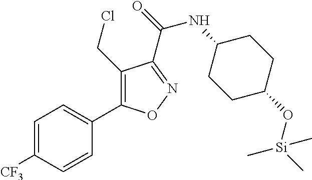

A solution of 4-(chloromethyl)-5-(4-(trifluoromethyl)phenyl)isoxazole-3-carbonyl chloride Example 1D (1.60 mmol, 500 mg) and triethylamine (1.60 mmol, 162 mg) in DCM (10 mL) was added to (1S, 4S)-4-(trimethylsilyloxy)cyclohexan-amine (1.93 mmol, 360 mg) in DCM (5 mL) and stirred at room temperature for 24 hours. The reaction mixture was then quenched with water and then DCM added and the solution poured through a hydrophobic frit. The organic layer was collected and evaporated to dryness. Taken onto the next step. MS (ESI) m/z (M+H⁺): 475.5.

### B: N-((1s,4s)-4-Hydroxycyclohexyl)-4-((isobutylamino)methyl)-5-(4-(trifluoromethyl)-phenyl)isoxazole-3-carboxamide

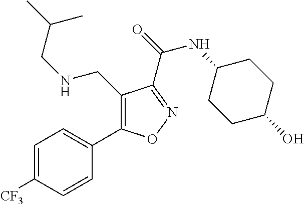

A solution of 4-(chloromethyl)-5-(4-(trifluoromethyl)phenyl)-N-((1s,4s)-4-(trimethylsilyloxy)cyclohexyl)isoxazole-3-carboxamide Example 7A (0.21 mmol, 100 mg), DIPEA (0.84 mmol, 0.14 mL, 109 mg) and isobutylamine (2.10 mmol, 0.21 mL, 154 mg) in acetonitrile (1 mL) were heated in the microwave at 165° C. for 900 s. The reaction mixture was diluted with DCM:water and poured through a hydrophobic frit. The crude product was added to a SCX column and was eluted with DCM, MeOH, 2M NH₃ in MeOH. Th product fractions were collected and evaporated to dryness. The resultant residue was dissolved in 1 mL of solvent (2:1:1 DMSO:water: aceto-nitrile), filtered and purified by preparative LCMS (basic). Product fractions evaporated to dryness to yield the title compound. MS (ESI) m/z (M+H⁺): 440.5.

The method of Example 7 was further used to prepare the following compounds using alternative amines instead of isobutylamine.

### EXAMPLE 8

### 8A: N-((1s,4s)-4-Hydroxycyclohexyl)-5-(4-(trifluoromethyl)phenyl)-4-((1,1,1-trifluoropropan-2-ylamino)methyl)isoxazole-3-carboxamide 2,2,2-trifluoroacetate

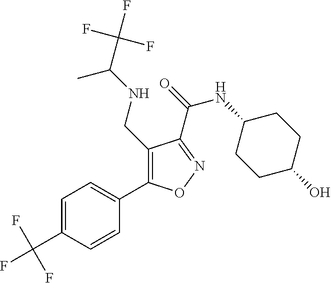

MS (ESI) m/z (M+H⁺): 480.5.

### 8B: N-((1s,4s)-4-Hydroxycyclohexyl)-4-((4-(trifluoromethyl)-benzylamino)methyl)-5-(4-(trifluoromethyl)-phenyl)isoxazole-3-carboxamide 2,2,2-trifluoroacetate MS (ESI) m/z (M+H⁺): 542.5.

### 8C: 4-((Benzylamino)methyl)-N-((1s,4s)-4-hydroxycyclohexyl)-5-(4-(trifluoromethyl)-phenyl)isoxazole-3-carboxamide 2,2,2-trifluoroacetate MS (ESI) m/z (M+H⁺): 474.5.

### 8D: N-((1s,4s)-4-Hydroxycyclohexyl)-4-(((tetrahydro-2H-pyran-4-yl)methylamino)methyl)-5-(4-(trifluoromethyl)-phenyl)isoxazole-3-carboxamide 2,2,2-trifluoroacetate

MS (ESI) m/z (M+H⁺): 482.5.

### 8E: N-((1s,4s)-4-Hydroxycyclohexyl)-4-((2-methoxy-2-methylpropylamino)methyl)-5-(4-(trifluoromethyl)-phenyl)isoxazole-3-carboxamide 2,2,2-trifluoroacetate MS (ESI) m/z (M+H⁺): 470.5.

### 8F: 4-((Ethyl(isopropyl)-amino)methyl)-N-((1s,4s)-4-hydroxycyclohexyl)-5-(4-(trifluoromethyl)-phenyl)isoxazole-3-carboxamide 2,2,2-trifluoroacetate

MS (ESI) m/z (M+H⁺): 454.5.

### 8G: N-((1 s,4s)-4-Hydroxycyclohexyl)-4-((isopropyl(methyl)amino)methyl)-5-(4-(trifluoromethyl)phenyl)isoxazole-3-carboxamide

MS (ESI) m/z (M+H⁺): 440.5.

### 8H: N-((1 s,4s)-4-Hydroxycyclohexyl)-4-((isopropylamino)methyl)-5-(4-(trifluoromethyl)-phenyl)isoxazole-3-carboxamide

MS (ESI) m/z (M+H⁺): 426.5.

### 8I: N-((1s,4s)-4-Hydroxycyclohexyl)-4-((tetrahydro-2H-pyran-4-ylamino)methyl)-5-(4-(trifluoromethyl)-phenyl)isoxazole-3-carboxamide

MS (ESI) m/z (M+H⁺): 468.5.

### EXAMPLE 9

### 5-(3-Fluoro-4-(trifluoromethyl)-phenyl)-N-((1S,3R)-3-hydroxycyclohexyl)-4-(((S)-1,1,1-trifluoropropan-2-ylamino)methyl)isoxazole-3-carboxamide

### A: Ethyl 5-(3-fluoro-4-(trifluoromethyl)phenyl)-4-methylisoxazole-3-carboxylate

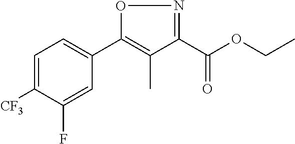

Tetrakis(triphenylphosphine)palladium (0) (0.12 mmol, 139 mg) was added to a suspension of 2-(3-fluoro-4-(trifluoromethyl)phenyl)-4,4,5,5-tetramethyl-1,3,2-dioxaborolane (1.21 mmol, 350 mg), ethyl 5-bromo-4-methylisoxazole-3-carboxylate (1.21 mmol, 282 mg) and potassium carbonate (2.41 mmol, 334 mg) in DME and water. The reaction mixture was sealed in a microwave vial and heated at 100° C. for 2 hours (oil bath). The reaction mixture was diluted with EtOAc and water, and extracted with EtOAc. The organics were combined, washed with brine and dried over MgSO₄, filtered and concentrated in vacuo before purifying on silica gel by SP4, using 25M column, 0-40% EtOAc:heptane, to give the desired product (51.2%). MS (ESI) m/z (M+H⁺): 318.5.

### B: Ethyl 4-(bromomethyl)-5-(3-fluoro-4-(trifluoromethyl)phenyl)isoxazole-3-carboxylate

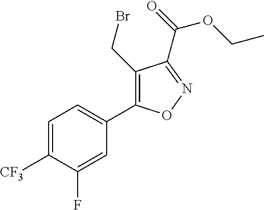

N-Bromosuccinimide (6.43 mmol, 1.15 g), benzoic peroxyanhydride (0.57 mmol, 0.13 g) and ethyl 5-(3-fluoro-4-(trifluoromethyl)phenyl)-4-methylisoxazole-3-carboxylate Example 9A (5.36 mmol, 1.7 g) were combined in a round bottomed flask with CCl₄ (50 mL) and refluxed at 88° C. for 4 hours. The reaction mixture was concentrated under vacuum and fresh CCl₄ was added and a further 0.5 eq of N-bromosuccinimide. The reaction mixture was again heated to 88° C. for 4 hours. The reaction mixture was concentrated under reduced pressure and the residue passed through a silica gel column eluting with 100% DCM to 3% MeOH:DCM on SP4 (column) to give the title compound (95%). MS (ESI) m/z (M+H⁺): 396.5.

### C: 5-(3-Fluoro-4-(trifluoromethyl)phenyl)-4-(hydroxymethyl)isoxazole-3-carboxylic acid

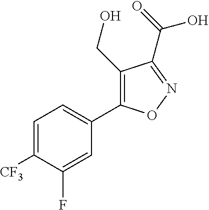

In 2×25 mL microwave vials was added ethyl 4-(bromomethyl)-5-(3-fluoro-4-(trifluoromethyl)phenyl)isoxazole-3-carboxylate Example 9B (18.05 mmol, 7.15 g) and trifluoroacetic acid in water (25%). The vials were sealed and heated in the microwave for 15 min at 150° C. The reaction mixture was diluted with water and filtered. The yellow crystalline solid was dried in the oven under vacuum for 25 hours. Taken onto next step. MS (ESI) m/z (M+H⁺): 306.5.

### D: 4-(Chloromethyl)-5-(3-fluoro-4-(trifluoromethyl)phenyl)isoxazole-3-carbonyl chloride

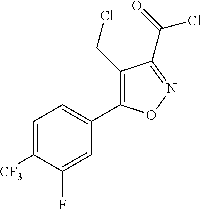

Thionyl chloride (0.94 mmol, 112 mg) was added to a suspension of 5-(3-fluoro-4-(trifluoromethyl)phenyl)-4-(hydroxymethyl)isoxazole-3-carboxylic acid Example 9C (0.39 mmol, 120 mg) in toluene (1 mL) and a drop of DMF. The suspension was heated at 90° C. for 4 hours. The reaction mixture was concentrated under reduced pressure to give a brown oil which was taken onto the next step. MS (ESI) m/z (M+H⁺): 343.5.

### E: 4-(Chloromethyl)-5-(3-fluoro-4-(trifluoromethyl)phenyl)-N-((1S,3R)-3-hydroxycyclohexyl)isoxazole-3-carboxamide

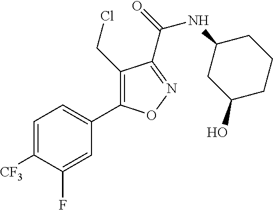

4-(Chloromethyl)-5-(3-fluoro-4-(trifluoromethyl)phenyl)isoxazole-3-carbonyl chloride Example 9D (0.30 mmol, 100 mg) was dissolved in DCM and the (1R,3S)-3-aminocyclohexanol (0.36 mmol, 42 mg) added. Triethylamine (0.61 mmol, 61 mg,) was added and the resulting solution stirred for 2 hours. Sodium bicarbonate solution was added and DCM separated using a hydrophobic frit. The DCM layer was concentrated under reduced pressure and the crude product passed through a silica gel column eluting with 0-10% MeOH in DCM to give the title compound. MS (ESI) m/z (M+H⁺): 421.5.

### F: 5-(3-Fluoro-4-(trifluoromethyl)phenyl)-N-((1S,3R)-3-hydroxycyclohexyl)-4-(((S)-1,1,1-trifluoropropan-2-ylamino)methyl)isoxazole-3-carboxamide

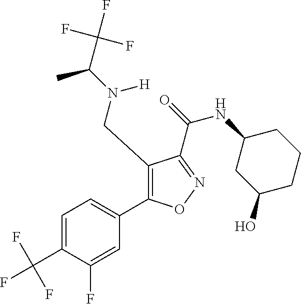

To a microwave vial was added 4-(chloromethyl)-5-(3-fluoro-4-(trifluoro-methyl)phenyl)-N-((1S,3R)-3-hydroxycyclohexyl)isoxazole-3-carboxamide Example 9E (0.23 mmol, 100 mg,) dissolved in acetonitrile and DIPEA (0.28 mmol 27 mg), followed by (S)-1,1,1-trifluoropropan-2-amine (0.48 mmol, 53 mg). The vial was sealed before heating in the microwave at 165° C. for 15 min. The reaction mixture was evaporated to dryness under reduced pressure and redissolved in DCM, washed with water and separated using hydrophobic frit. The organics were then evaporated under reduced pressure and redissolved in DMSO and purified by HPLC (acidic) to yield the title compound. MS (ESI) m/z (M+H⁺): 498.5.

The method of Example 9 was further used to prepare the following compounds using alternative amines instead of (S)-1,1,1-trifluoropropan-2-amine.

### EXAMPLE 10

### 10A: 4-((Ethyl(isopropyl)amino)methyl)-5-(3-fluoro-4-(trifluoromethyl)phenyl)-N-((1S,3R)-3-hydroxycyclohexyl)isoxazole-3-carboxamide

MS (ESI) m/z (M+H⁺): 472.5.

### 10B: 4-((2-Cyclopropylethylamino)methyl)-5-(3-fluoro-4-(trifluoromethyl)phenyl)-N-((1S,3R)-3-hydroxycyclohexyl)isoxazole-3-carboxamide

MS (ESI) m/z (M+H⁺): 470.5.

### 10C: 5-(3-Fluoro-4-(trifluoromethyl)phenyl)-N-((1S,3R)-3-hydroxycyclohexyl)-4-((isobutylamino)methyl)isoxazole-3-carboxamide

MS (ESI) m/z (M+H⁺): 458.5.

### 10D: 4-((Cyclopropylmethylamino)methyl)-5-(3-fluoro-4-(trifluoromethyl)phenyl)-N-((1S,3R)-3-hydroxycyclohexyl)isoxazole-3-carboxamide

MS (ESI) m/z (M+H⁺): 456.5.

### 10E: 4-((Cyclobutylmethylamino)methyl)-5-(3-fluoro-4-(trifluoromethyl)phenyl)-N-((1S,3R)-3-hydroxycyclohexyl)isoxazole-3-carboxamide

MS (ESI) m/z (M+H⁺): 470.5.

### 10F: 5-(3-Fluoro-4-(trifluoromethyl)phenyl)-4-4-(((R)-1-hydroxy-3-methylbutan-2-ylamino)methyl)-N-((1S,3R)-3-hydroxycyclohexyl)isoxazole-3-carboxamide

MS (ESI) m/z (M+H⁺): 488.5.

### 10G: 5-(3-Fluoro-4-(trifluoromethyl)phenyl)-4-((3-hydroxy-2,2-dimethylpropylamino)methyl)-N-((1S,3R)-3-hydroxycyclohexyl)isoxazole-3-carboxamide

MS (ESI) m/z (M+H⁺): 488.5.

### 10H: 5-(3-Fluoro-4-(trifluoromethyl)phenyl)-N-((1S,3R)-3-hydroxycyclohexyl)-4-(((2-methoxyethyl)(methyl)amino)methyl)isoxazole-3-carboxamide

MS (ESI) m/z (M+H⁺): 473.5.

### 10I: 5-(3-Fluoro-4-(trifluoromethyl)phenyl)-N-((1S,3R)-3-hydroxycyclohexyl)-4-(((tetrahydro-2H-pyran-4-yl)methylamino)methyl)isoxazole-3-carboxamide

MS (ESI) m/z (M+H⁺): 473.5.

### 10J: 4-((Cyclobutylamino)methyl)-5-(3-fluoro-4-(trifluoromethyl)phenyl)-N-((1S,3R)-3-hydroxycyclohexyl)isoxazole-3-carboxamide

MS (ESI) m/z (M+H⁺): 500.5.

### 10K: 5-(3-Fluoro-4-(trifluoromethyl)phenyl)-N-((1S,3R)-3-hydroxycyclohexyl)-4-((2-methoxyethylamino)methyl)isoxazole-3-carboxamide

MS (ESI) m/z (M+H⁺): 460.5.

### 10L: 5-(3-Fluoro-4-(trifluoromethyl)phenyl)-N-((1S,3R)-3-hydroxycyclohexyl)-4-((2-(piperidin-1-yl)ethylamino)methyl)isoxazole-3-carboxamide

MS (ESI) m/z (M+H⁺): 513.5.

### 10M: 5-(3-Fluoro-4-(trifluoromethyl)phenyl)-N-((1S,3R)-3-hydroxycyclohexyl)-4-((methyl(1-methylpiperidin-4-yl)amino)methyl)isoxazole-3-carboxamide

MS (ESI) m/z (M+H⁺): 513.5.

### 10N: 5-(3-Fluoro-4-(trifluoromethyl)phenyl)-N-((1S,3R)-3-hydroxycyclohexyl)-4-(((2-hydroxyethyl)(isopropyl)amino)methyl)isoxazole-3-carboxamide

MS (ESI) m/z (M+H⁺): 488.5.

### EXAMPLE 11

### N-((1s,4s)-4-Hydroxycyclohexyl)-4-((isopropylamino)methyl)-5-(4-(trifluoromethyl)-phenyl)isoxazole-3-carboxamide

### A: Ethyl 4-((isopropylamino)methyl)-5-(4-(trifluoromethyl)phenyl)isoxazole-3-carboxylate

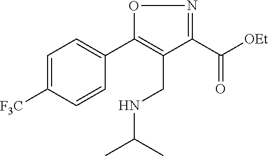

Ethyl 4-(bromomethyl)-5-(4-(trifluoromethyl)phenyl)isoxazole-3-carboxylate Example 1B (1.33 mmol, 500 mg) was dissolved in dichloromethane and triethylamine (2.65 mmol, 268 mg) added. Cooled to 0-5° C. and isopropylamine (1.33 mmol, 38 mg) added dropwise. Stirred overnight then partitioned between saturated sodium bicarbonate solution and EtOAc. The organic phase was dried over Na₂SO₄ and purified by column chromatography eluting with 5% MeOH:DCM to give the title compound. MS (ESI) m/z (M+H⁺): 357.5.

### B: 4-((Isopropylamino)methyl)-5-(4-(trifluoromethyl)phenyl)isoxazole-3-carboxylic acid

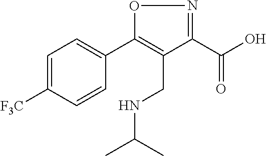

Ethyl 4-((isopropylamino)methyl)-5-(4-(trifluoromethyl)phenyl)isoxazole-3-carboxylate Example 11A (0.33 mmo, 118 mg) dissolved in THF (1 mL) and LiOH (1M, 0.49 mL, 0.49 mmol) added. Stirred for 2 hours, acidified with 1M HCl and the resulting solid filtered. The solid was then dried under vacuum to give the title compound (64 mg, 59%). MS (ESI) m/z (M+H⁺): 329.5.

### C: N-((1S,4S)-4-Hydroxycyclohexyl)-4-((isopropylamino)methyl)-5-(4-(trifluoro-methyl)phenyl)isoxazole-3-carboxamide

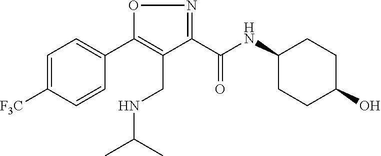

4-((Isopropylamino)methyl)-5-(4-(trifluoromethyl)phenyl)isoxazole-3-carboxylic acid Example 11B (0.08 mmol, 27 mg), (1S,4S)-4-aminocyclohexanol (0.08 mmol, 9.5 mg), HATU (0.11 mmol, 41 mg) and triethylamine (0.66 mmol, 0.089 mL) were stirred at room temperature in DCM (1 mL) for 2 hours. The reaction mixture washed with saturated sodium bicarbonate solution and purified by silica gel column chromatography eluting with 0-100% DCM:EtOAc to give the title compound (18 mg, 51%). MS (ESI) m/z (M+H⁺): 426.5.

The method of Example 11 was further used to prepare the following compounds using alternative amines instead of (1S,4S)-4-aminocyclohexanol.

### EXAMPLE 12

### 12A: (S)-4-((Isopropylamino)methyl)-N-(3-methylbutan-2-yl)-5-(4-(trifluoromethyl)phenyl)isoxazole-3-carboxamide

MS (ESI) m/z (M+H⁺): 398.5.

### 12B: (4-Hydroxypiperidin-1-yl)(4-((isopropylamino)methyl)-5-(4-(trifluoromethyl)phenyl)isoxazol-3-yl)methanone

MS (ESI) m/z (M+H⁺): 412.5.

### 12C: N-Cyclopentyl-4-((isopropylamino)methyl)-5-(4-(trifluoromethyl)-phenyl)isoxazole-3-carboxamide

MS (ESI) m/z (M+H⁺): 396.5.

### 12D: (S)—N-sec-Butyl-4-((isopropylamino)methyl)-5-(4-(trifluoromethyl)-phenyl)isoxazole-3-carboxamide

MS (ESI) m/z (M+H⁺): 384.5.

### 12E: N-(4-Hydroxyphenyl)-4-((isopropylamino)methyl)-5-(4-(trifluoromethyl)phenyl)isoxazole-3-carboxamide

MS (ESI) m/z (M+H⁺): 420.5.

### 12F: N-(3-(Hydroxymethyl)phenyl)-4-((isopropylamino)methyl)-5-(4-(trifluoromethyl)phenyl)isoxazole-3-carboxamide

MS (ESI) m/z (M+H⁺): 424.5.

### 12G: (R)-4-((Isopropylamino)methyl)-N-(3-methylbutan-2-yl)-5-(4-(trifluoromethyl)phenyl)isoxazole-3-carboxamide

MS (ESI) m/z (M+H⁺): 398.5.

### 12H: N-(4-(Hydroxymethyl)phenyl)-4-((isopropylamino)methyl)-5-(4-(trifluoromethyl)phenyl)isoxazole-3-carboxamide

MS (ESI) m/z (M+H⁺): 434.5.

### 12I: 4-((Isopropylamino)methyl)-N-propyl-5-(4-(trifluoromethyl)phenyl)isoxazole-3-carboxamide

MS (ESI) m/z (M+H⁺): 370.5.

### 12J: (R)—N-(1-Hydroxy-3-methylbutan-2-yl)-4-((isopropylamino)methyl)-5-(4-(trifluoromethyl)phenyl)isoxazole-3-carboxamide

MS (ESI) m/z (M+H⁺): 414.5.

### 12K: (4-((Isopropylamino)methyl)-5-(4-(trifluoromethyl)phenyl)isoxazol-3-yl)(piperidin-1-yl)methanone

MS (ESI) m/z (M+H⁺): 396.5.

### 12L: N-Isobutyl-4-((isopropylamino)methyl)-5-(4-(trifluoromethyl)phenyl)isoxazole-3-carboxamide

MS (ESI) m/z (M+H⁺): 384.5.

### 12M: N-(1-Cyclopropyl-3-hydroxypropyl)-4-((isopropylamino)methyl)-5-(4-(trifluoromethyl)phenyl)isoxazole-3-carboxamide

MS (ESI) m/z (M+H⁺): 426.5.

### 12N: N-((1R,2S)-2-(Hydroxymethyl)cyclohexyl)-4-((isopropylamino)methyl)-5-(4-(trifluoromethyl)phenyl)isoxazole-3-carboxamide

MS (ESI) m/z (M+H⁺): 440.5.

### EXAMPLE 13

### N-Cyclopentyl-4-((isopropyl(methyl)amino)methyl)-5-(4-(trifluoromethyl)phenyl)isoxazole-3-carboxamide

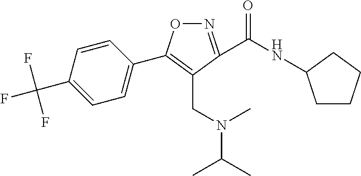

N-Cyclopentyl-4-((isopropylamino)methyl)-5-(4-(trifluoromethyl)phenyl)-isoxazole-3-carboxamide Example 12C (0.17 mmol, 50 mg), formaldehyde (1.26 mmol, 0.095 mL), sodium triacetoxyborohydride (0.38 mmol, 80 mg), acetic acid (2 drops) and acetonitrile (3 mL) were placed in a microwave vial and treated with microwaves at 175° C. for 2100 s. The reaction mixture was partitioned between water and DCM and the organic layer collected. The organic were concentrated in vacuo and the residue purified by SCX column yielding the title compound (26 mg, 50%). MS (ESI) m/z (M+H⁺): 410.5.

The method of Example 13 was further used to prepare the following compounds using alternative aldehydes instead of formaldehyde.

### EXAMPLE 14

### 14A: N-Cyclopentyl-4-((isobutyl(isopropyl)amino)methyl)-5-(4-(trifluoromethyl)phenyl)isoxazole-3-carboxamide

MS (ESI) m/z (M+H⁺): 452.5.

### 14B: N-Cyclopentyl-4-((isopropyl(propyl)amino)methyl)-5-(4-(trifluoromethyl)phenyl)isoxazole-3-carboxamide

MS (ESI) m/z (M+H⁺): 438.5.

### 14C: N-Cyclopentyl-4-(((cyclopropylmethyl)(isopropyl)amino)methyl)-5-(4-(trifluoromethyl)phenyl)isoxazole-3-carboxamide

MS (ESI) m/z (M+H⁺): 450.5.

### 14D: N-Cyclopentyl-4-((ethyl(isopropyl)amino)methyl)-5-(4-(trifluoromethyl)phenyl)isoxazole-3-carboxamide

MS (ESI) m/z (M+H⁺): 424.5.

### 14E: N-Cyclopentyl-4-((isopropyl(3,3,3-trifluoropropyl)amino)methyl)-5-(4-(trifluoromethyl)phenyl)isoxazole-3-carboxamide

MS (ESI) m/z (M+H⁺): 492.5.

### EXAMPLE 15

### N-Cyclopentyl-4-((N-isopropyl methylsulfonamido)methyl)-5-(4-(trifluoromethyl)phenyl)isoxazole-3-carboxamide

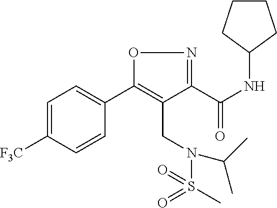

N-Cyclopentyl-4-((isopropylamino)methyl)-5-(4-(trifluoromethyl)phenyl)-isoxazole-3-carboxamide Example 12C (0.13 mmol, 50 mg), methanesulphonyl chloride (0.38 mmol, 0.03 mL,) and triethylamine (0.38 mmol, 0.05 mL) were stirred in DCM (1 mL) for 1 hour. The reaction mixture washed with water and purified by silica gel column chromatography eluting with 0-30% EtOAc:DCM yielding the title compound (25 mg, 42%). MS (ESI) m/z (M+H⁺): 474.5.

The method of Example 15 was further used to prepare the following compounds using alternative sulphonyl chlorides or acid chlorides instead of methanesulphonyl chloride

### EXAMPLE 16

### 16A: N-Cyclopentyl-4-((N-isopropylcyclopropanesulfonamido)methyl)-5-(4-(trifluoromethyl)phenyl)isoxazole-3-carboxamide

MS (ESI) m/z (M+H⁺): 500.5.

### 16B: N-Cyclopentyl-4-((2,2,2-trifluoro-N-isopropylacetamido)methyl)-5-(4-(trifluoromethyl)phenyl)isoxazole-3-carboxamide

MS (ESI) m/z (M+H⁺): 492.5.

### 16C: N-Cyclopentyl-4-((N-isopropyl-2-methylbutanamido)methyl)-5-(4-(trifluoromethyl)phenyl)isoxazole-3-carboxamide

MS (ESI) m/z (M+H⁺): 480.5.

### 16D: N-Cyclopentyl-4-((N-isopropylisobutyramido)methyl)-5-(4-(trifluoromethyl)phenyl)isoxazole-3-carboxamide

MS (ESI) m/z (M+H⁺): 466.5.

### 16E: N-Cyclopentyl-4-((N-isopropylcyclopropanecarboxamido)methyl)-5-(4-(trifluoromethyl)phenyl)isoxazole-3-carboxamide

MS (ESI) m/z (M+H⁺): 464.5.

### 16F: N-Cyclopentyl-4-((N-isopropylpropionamido)methyl)-5-(4-(trifluoromethyl)phenyl)isoxazole-3-carboxamide

MS (ESI) m/z (M+H⁺): 452.5.

### EXAMPLE 17

### 4-((2-Cyclopropylethylamino)methyl)-5-(3-fluoro-4-(trifluoromethyl)phenyl)-N-((1R,3S)-3-hydroxycyclohexyl)isoxazole-3-carboxamide

### A: 4-(Chloromethyl)-5-(3-fluoro-4-(trifluoromethyl)phenyl)-N-((1R,3S)-3-hydroxycyclohexyl)isoxazole-3-carboxamide

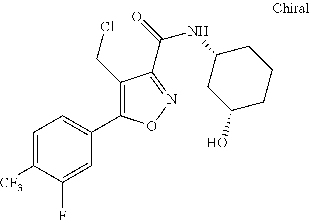

4-(Chloromethyl)-5-(3-fluoro-4-(trifluoromethyl)phenyl)isoxazole-3-carbonyl chloride Example 9D (1.47 mmol, 500 mg) was dissolved in DCM (10 mL) and the (1S,3R)-3-aminocyclohexanol (1.47 mmol, 169 mg) added. Triethylamine (2.94 mmol, 297 mg) was added and the resulting solution stirred for 2 hours. Sodium bicarbonate solution was added and the DCM layer separated using a hydrophobic frit. The organics were concentrated under reduced pressure and the crude product passed through a silica gel column eluting with 0-10% MeOH:DCM to give the title compound.

MS (ESI) m/z (M+H⁺): 421.5.

### B: 4-((2-Cyclopropylethylamino)methyl)-5-(3-fluoro-4-(trifluoromethyl)phenyl)-N-((1R,3S)-3-hydroxycyclohexyl)isoxazole-3-carboxamide

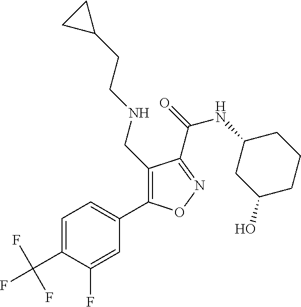

To a microwave vial was added 4-(chloromethyl)-5-(3-fluoro-4-(trifluoro-methyl)phenyl)-N-((1R,3S)-3-hydroxycyclohexyl)isoxazole-3-carboxamide Example 17A (0.23 mmol, 100 mg) dissolved in acetonitrile and DIPEA (0.47 mmol, 61 mg), followed by 2-cyclopropylethanamine (0.23 mmol, 20 mg). The vial was sealed before heating in the microwave at 165° C. for 15 min. The reaction mixture was evaporated to dryness under reduced pressure and redissolved in DCM, washed with water and separated using a hydrophobic frit. The organics were then evaporated under reduced pressure and redissolved in DMSO and purified by HPLC (acidic) to yield the title compound. MS (ESI) m/z (M+H⁺): 470.5.

The method of Example 17 was further used to prepare the following compounds using alternative amines instead of 2-cyclopropylethanamine.

### EXAMPLE 18

### 18A: 4-((Ethyl(isopropyl)amino)methyl)-5-(3-fluoro-4-(trifluoromethyl)phenyl)-N-((1R,3S)-3-hydroxycyclohexyl)isoxazole-3-carboxamide

MS (ESI) m/z (M+H⁺): 472.5.

### 18B: 4-((Cyclopropylamino)methyl)-5-(3-fluoro-4-(trifluoromethyl)phenyl)-N-((1R,3S)-3-hydroxycyclohexyl)isoxazole-3-carboxamide

MS (ESI) m/z (M+H⁺): 442.5.

### 18C: 5-(3-Fluoro-4-(trifluoromethyl)phenyl)-N-((1R,3S)-3-hydroxycyclohexyl)-4-((isobutylamino)methyl)isoxazole-3-carboxamide

MS (ESI) m/z (M+H⁺): 458.5.

### 18D: 4-((Cyclobutylmethylamino)methyl)-5-(3-fluoro-4-(trifluoromethyl)phenyl)-N-((1R,3S)-3-hydroxycyclohexyl)isoxazole-3-carboxamide

MS (ESI) m/z (M+H⁺): 470.5.

### 18E: 5-(3-Fluoro-4-(trifluoromethyl)phenyl)-4-(((R)-1-hydroxy-3-methylbutan-2-ylamino)methyl)-N-((1R,3S)-3-hydroxycyclohexyl)isoxazole-3-carboxamide

MS (ESI) m/z (M+H⁺): 488.5.

### 18F: 5-(3-Fluoro-4-(trifluoromethyl)phenyl)-N-((1R,3S)-3-hydroxycyclohexyl)-4-(((tetrahydro-2H-pyran-4-yl)methylamino)methyl)isoxazole-3-carboxamide

MS (ESI) m/z (M+H⁺): 500.5.

### 18G: 5-(3-Fluoro-4-(trifluoromethyl)phenyl)-N-((1R,3S)-3-hydroxycyclohexyl)-4-(((2-methoxyethyl)(methyl)amino)methyl)isoxazole-3-carboxamide

MS (ESI) m/z (M+H⁺): 474.5.

### 18H: 4-((Cyclobutylamino)methyl)-5-(3-fluoro-4-(trifluoromethyl)phenyl)-N-((1R,3S)-3-hydroxycyclohexyl)isoxazole-3-carboxamide

MS (ESI) m/z (M+H⁺): 456.5.

### 18I: 5-(3-Fluoro-4-(trifluoromethyl)phenyl)-N-((1R,3S)-3-hydroxycyclohexyl)-4-(((R)-tetrahydrofuran-3-ylamino)methyl)isoxazole-3-carboxamide

MS (ESI) m/z (M+H⁺): 472.5.

### 18J: 5-(3-Fluoro-4-(trifluoromethyl)phenyl)-N-((1R,3S)-3-hydroxycyclohexyl)-4-((2-methoxyethylamino)methyl)isoxazole-3-carboxamide

MS (ESI) m/z (M+H⁺): 460.5.

### 18K: 5-(3-Fluoro-4-(trifluoromethyl)phenyl)-N-((1R,3S)-3-hydroxycyclohexyl)-4-((methyl(1-methylpiperidin-4-yl)amino)methyl)isoxazole-3-carboxamide

MS (ESI) m/z (M+H⁺): 513.5.

### EXAMPLE 19

### N-((1R,3S)-3-Hydroxycyclohexyl)-4-(hydroxymethyl)-5-(4-(trifluoromethyl)phenyl)isoxazole-3-carboxamide

### A: Ethyl 4-(hydroxymethyl)-5-(4-(trifluoromethyl)phenyl)isoxazole-3-carboxylate

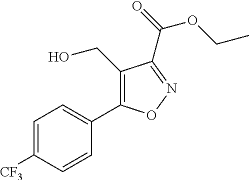

In a 20 mL microwave vial was added ethyl 4-(bromomethyl)-5-(4-(trifluoro-methyl)phenyl)isoxazole-3-carboxylate Example 1B (3.97 mmol, 1.5 g) and trifluoroacetic acid in water (25% v/v TFA:water=15 mL) added. The suspension was heated in the microwave at 150° C. for 15 min. before diluting with water. The resultant precipitate was filtered off and dried in an oven at 50° C. under vacuum overnight. The crude product was added to a silica gel column and was eluted with 5% MeOH:DCM. ¹H NMR showed 2:1 mixture of title compound and Example 1C. Taken onto the next step.

### B: N-((1R,3S)-3-Hydroxycyclohexyl)-4-(hydroxymethyl)-5-(4-(trifluoromethyl)phenyl)isoxazole-3-carboxamide

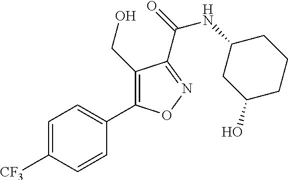

Ethyl 4-(hydroxymethyl)-5-(4-(trifluoromethyl)phenyl)isoxazole-3-carboxylate Example 19A (1.58 mmol, 500 mg) was added to a microwave vial containing acetonitrile. Triethylamine (3.17 mmol, 320 mg) was then added followed by (1S,3R)-3-aminocyclohexanol (1.58 mmol, 182 mg) and the resulting mixture heated at 150° C. for 10 min. Acetonitrile was removed from all the sample under reduced pressure and the crude residue taken up in DCM, washed with water and separated using a hydrophibic frit. The DCM layer was evaporated to dryness and the crude residue purified by column chromatography using 0-10% MeOH:DCM, 12M silica cartridge on the SP4 to give the desired product. MS (ESI) m/z (M+H⁺): 385.5.

The method of Example 19 was further used to prepare the following compounds using alternative amines instead of (1S,3R)-3-aminocyclohexanol.

### EXAMPLE 20

### 20A: (S)-4-(Hydroxymethyl)-N-(3-methylbutan-2-yl)-5-(4-(trifluoromethyl)phenyl)isoxazole-3-carboxamide

MS (ESI) m/z (M+H⁺): 357.5.

### 20B: N-Cyclopentyl-4-(hydroxymethyl)-5-(4-(trifluoromethyl)phenyl)isoxazole-3-carboxamide

MS (ESI) m/z (M+H⁺): 355.5.

### 20C: 4-(Hydroxymethyl)-N-((1S,2R)-2-(hydroxymethyl)cyclohexyl)-5-(4-(trifluoromethyl)phenyl)isoxazole-3-carboxamide

MS (ESI) m/z (M+H⁺): 399.5.

### 20D: (R)—N-(1-Hydroxybutan-2-yl)-4-(hydroxymethyl)-5-(4-(trifluoromethyl)phenyl)isoxazole-3-carboxamide

MS (ESI) m/z (M+H⁺): 359.5.

### 20E: N-((1S,4S)-4-Hydroxycyclohexyl)-4-(hydroxymethyl)-5-(4-(trifluoromethyl)phenyl)isoxazole-3-carboxamide

MS (ESI) m/z (M+H⁺): 385.5.

### 20F: 4-(Hydroxymethyl)-N-(tetrahydro-2H-pyran-4-yl)-5-(4-(trifluoromethyl)phenyl)isoxazole-3-carboxamide

MS (ESI) m/z (M+H⁺): 371.5.

### EXAMPLE 21

### (S)-5-(3-Fluoro-4-(trifluoromethyl)phenyl)-4-(hydroxymethyl)-N-(3-methylbutan-2-yl)isoxazole-3-carboxamide

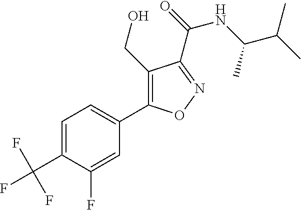

5-(3-Fluoro-4-(trifluoromethyl)phenyl)-4-(hydroxymethyl)isoxazole-3-carboxylic acid Example 9C (0.33 mmol, 100 mg), (S)-3-methylbutan-2-amine (0.33 mmol, 29 mg) and triethylamine (0.66 mmol, 66.7 mg) were added to a vial containing DCM. The solution was stirred for 5 min. before the addition of cyclophos/PPA 50% in EtOAc. The reaction mixture was stirred for 1.5 hours before quenching with the addition of sodium bicarbonate. The DCM layer was separated using a hydrophobic frit, and the DCM removed under reduced pressure. The residue was purified by acidic HPLC, to give the desired product. MS (ESI) m/z (M+H⁺): 375.5.

The method of Example 21 was further used to prepare the following compounds using alternative amines instead of (S)-3-methylbutan-2-amine.

### EXAMPLE 22

### 22A: N-Cyclopentyl-5-(3-fluoro-4-(trifluoromethyl)phenyl)-4-(hydroxymethyl)isoxazole-3-carboxamide

MS (ESI) m/z (M+H⁺): 373.5.

### 22B: (R)-5-(3-Fluoro-4-(trifluoromethyl)phenyl)-N-(1-hydroxybutan-2-yl)-4-(hydroxymethyl)isoxazole-3-carboxamide

MS (ESI) m/z (M+H⁺): 377.5.

### 22C: 5-(3-Fluoro-4-(trifluoromethyl)phenyl)-N-((1s,4s)-4-hydroxycyclohexyl)-4-(hydroxymethyl)isoxazole-3-carboxamide

MS (ESI) m/z (M+H⁺): 403.5.

### 22D: (R)-5-(3-Fluoro-4-(trifluoromethyl)phenyl)-4-(hydroxymethyl)-N-(3-methylbutan-2-yl)isoxazole-3-carboxamide

MS (ESI) m/z (M+H⁺): 375.5.

### 22E: N-(3,3-Difluorocyclobutyl)-5-(3-fluoro-4-(trifluoromethyl)phenyl)-4-(hydroxymethyl)isoxazole-3-carboxamide

MS (ESI) m/z (M+H⁺): 395.5.

### EXAMPLE 23

### 4-((Cyclobutylamino)methyl)-N-cyclopentyl-5-(4-(trifluoromethyl)phenyl)isoxazole-3-carboxamide

### A: Ethyl 2,4-dioxo-4-(4-(trifluoromethyl)phenyl)butanoate

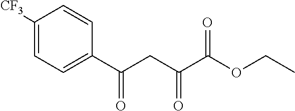

Diethyl oxalate (266 mmol, 36.1 mL, 38.8 g) was slowly added to a solution of sodium ethanolate (266 mmol, 99 mL, 86 g) in (dry) toluene (415 mL) at <10° C. under nitrogen. Once added the resulting orange solution was removed from the ice bath and stirred for 30 min. Then 1-(4-(trifluoromethyl)phenyl)ethanone (266 mmol, 50 g) was added portionwise. A very thick orange/brown suspension formed which was stirred overnight (mechanical stirring required) (NB ˜50 mL toluene added to aid stirring). The suspension was filtered and the resulting solid washed with toluene to yield a yellow solid. This was partitioned between 1000 mL of water containing 200 mL of 2N HCl and diethyl ether. Once dissolved the organic layer was separated and washed with brine, dried over Na₂Sa₄ and taken to dryness to give a yellow oil which solidified on cooling to give the title compound as a low melting yellow/orange solid (66 g, 86%). MS (ESI) m/z (M+H⁺): 289.5.

### B: ethyl 5-(4-(trifluoromethyl)phenyl)isoxazole-3-carboxylate

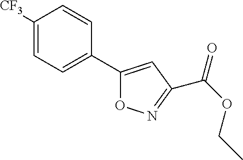

To a solution of ethyl 2,4-dioxo-4-(4-(trifluoromethyl)phenyl)butanoate Example 23A (229 mmol, 66 g) in ethanol with hydroxylamine hydrochloride (19.10 g) was refluxed for 3 hours. The mixture was allowed to cool and stirred for 1 hour and the resultant fluffy white solid filtered, washed with ethanol and dried in a oven at 45° C. under vacuum to give the title compound as a white solid, 51.2 g. MS (ESI) m/z (M+H⁺): 286.5.

### C: ethyl 4-bromo-5-(4-(trifluoromethyl)phenyl)isoxazole-3-carboxylate

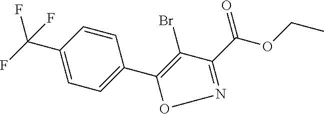

Ethyl 5-(4-(trifluoromethyl)phenyl)isoxazole-3-carboxylate Example 23B (17.5 mmol, 5.0 g), N-bromosuccinimide (21.0 mmol, 3.74 g), TFA 15 mL were combined and treated with microwaves at 150° C. for 20 mins. The reaction mixture was poured onto water and the resulting white precipitate collected by filtration to yield the desired product (6.1 g, 96%). MS (ESI) m/z (M+H⁺): 365.5.

### D: 4-bromo-N-cyclopentyl-5-(4-(trifluoromethyl)phenyl)isoxazole-3-carboxamide

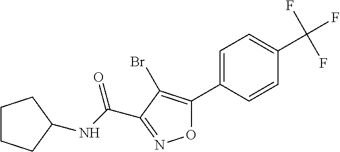

Ethyl 4-bromo-5-(4-(trifluoromethyl)phenyl)isoxazole-3-carboxylate Example 23C (2.75 mmol, 1.0 g) and cyclopentylamine (3.31 mmol, 281 mg) combined and treated with microwaves at 180° C. for 30 mins. The colour changed from clear to brown. The reaction mixture was poured onto water and the resulting precipitate filtered. The solid was passed through a silica gel column eluting with heptane to 40% EtOAc:heptane to give the title compound. MS (ESI) m/z (M+H⁺): 404.5.

### E: N-cyclopentyl-4-formyl-5-(4-(trifluoromethyl)phenyl)isoxazole-3-carboxamide

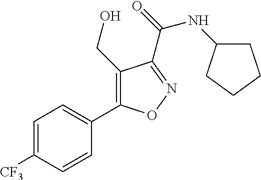

4-Bromo-N-cyclopentyl-5-(4-(trifluoromethyl)phenyl)isoxazole-3-carboxamide Example 23D (0.248 mmol, 100 mg) in THF (1 mL) was cooled in an acetone/CO₂ bath and ⁿBuLi (0.55 mmol, 0.22 mL) added slowly. After stirring for 1.5 hours, DMF (0.25 mmol) was added and the reaction stirred for 2 hours with acetone/CO₂ cooling. Water was added and the reaction allowed to reach room temperature. Diethyl ether was added and the organic phase separated. The organics were dried over MgSO₄ and concentrated in vacuo. Column chromatography on silica gel eluting with heptane to 30% EtOAc:heptane yielded the title compound (62 mg, 71%). MS (ESI) m/z (M+H⁺): 353.5.

### F: 4-((cyclobutylamino)methyl)-N-cyclopentyl-5-(4-(trifluoromethyl)phenyl)isoxazole-3-carboxamide

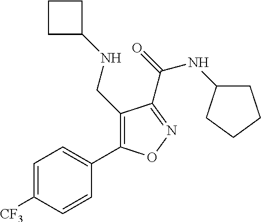

N-Cyclopentyl-4-formyl-5-(4-(trifluoromethyl)phenyl)isoxazole-3-carboxamide Example 23E (0.085 mmol 30 mg), cyclobutylamine (0.85 mmol, 61 mg), sodium triacetoxyborohydride (0.26 mmol, 54 mg), acetic acid (2 drops) and acetonitrile (0.5 mL) were combined in a vial and treated with microwaves at 175° C. for 2100 s. The reaction mixture was partitioned between DCM and water and the organic phase collected and evaporated to dryness. The residue was purified by column chromatography on silica gel eluting with 50% DCM:EtOAc) yielded the title compound (7 mg, 20%). MS (ESI) m/z (M+H⁺): 408.5.

The method of Example 23 was further used to prepare the following compound using alternative amine instead of cyclobutylamine

### EXAMPLE 24

### N-cyclopentyl-4-((2-hydroxyethylamino)methyl)-5-(4-(trifluoromethyl)phenyl)-isoxazole-3-carboxamide

MS (ESI) m/z (M+H⁺): 398.5.

### EXAMPLE 25

### N-cyclopentyl-4-(2-hydroxypropan-2-yl)-5-(4-(trifluoromethyl)phenyl)isoxazole-3-carboxamide

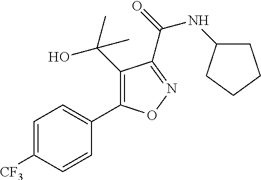

4-Bromo-N-cyclopentyl-5-(4-(trifluoromethyl)phenyl)isoxazole-3-carboxamide Example 23D (0.62 mmol, 250 mg) in THF (2.5 mL) was cooled in an acetone/CO₂ bath and nBuLi (1.36 mmol, 0.62 mL) added slowly. After stirring for 0.5 hours, acetone (3.10 mmol, 0.23 mL, 180 mg) was added and the reaction stirred for 2 hours with acetone/CO₂ cooling. Water was added and the reaction allowed to reach room temperature. Diethyl ether was added and the organic phase separated. The organics dried over MgSO₄ and concentrated in vacuo. The residue was passed through a silica gel column eluting with 50% DCM:EtOAc to give the title compound (15 mg, 6%). MS (ESI) m/z (M+H⁺): 383.5.

The method of Example 25 was further used to prepare the following compound using an alternative ketone instead of acetone.

### EXAMPLE 26

### N-cyclopentyl-4-(1-hydroxyethyl)-5-(4-(trifluoromethyl)phenyl)isoxazole-3-carboxamide

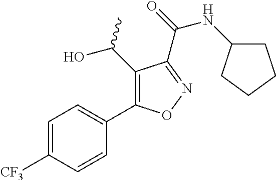

MS (ESI) m/z (M+H⁺): 369.5.

### EXAMPLE 27

### Vanilloid Receptor Binding Assay

Test compounds were prepared as stock solution in dimethylsulfoxide and tested for activity over several log units (ranging 100 μM-100 μM). Compounds were further diluted in assay buffer as necessary for IC₅₀ determination.

Chinese hamster ovary cells expressing human VR1 were grown in DMEM/F12 50/50 Mix (Mediatech, Inc., Herndon, Va., USA), supplemented with 10% FetalClone II (Hyclone, Logan, Utah, USA), 1% GlutaMax (Invitrogen Corp., Carlsbad, Calif., USA), 1% Pen/Strep (Mediatech) and 0.4 mg/ml G418 (Mediatech). The day before the assay, cells were seeded into 384-well tissue culture-treated black plates with clear bottoms (Corning, Inc., Corning, N.Y., USA), at 10,000 viable cells/well in 50 μl/well of medium containing no G418.

On the day of the assay, which is the FLIPR® Calcium 3 Assay commercially available from Molecular Devices Corp., Sunnyvale, Calif. USA, the plating medium was removed and replaced with 25 μl/well 1× Calcium 3 Assay kit dye, prepared in VR1 Buffer (160 mM NaCl, 4.5 mM KCl, 10 mM HEPES, 10 mM Glucose, 2 mM CaCl₂, 1 mM MgCl₂ and 0.5 mM Probenecid). After 1 hour incubation at room temperature, the plates were loaded into the FLIPR (Molecular Devices, Corp.), which adds 12.5 μl of test compound in VR1 Buffer containing 4% dimethylsulfoxide and reads the subsequent change in the fluorescence of the cells to monitor agonist activity. Ten minutes after compound addition, the plates were reloaded into the FLIPR, which adds 12.5 μl of 30 nM capsaicin in VR1 Buffer and reads the subsequent change in the fluorescence of the cells to monitor antagonist activity. In this way, the same assay was used to assess both the agonist activity and antagonist activity of test compounds.

Typical IC₅₀ values measured in the in vitro assay described above for the compounds of the invention are 10 μM or less. For several embodiments of the invention the IC₅₀ was found to be below 100 nM.

1-10. (canceled)

11. An isoxazole-3-carboxamide derivative having the general Formula I 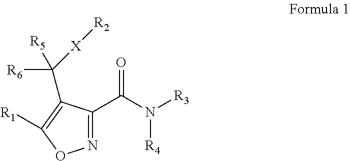
wherein
R₁ is phenyl or pyridyl, optionally substituted by 1-3 substituents selected from halogen, (C₁₋₄alkyl and (C₁₋₄)alkyloxy, the alkyl and alkyloxy group being optionally substituted with halogen;
X is O or NR₇;
R₂ is H or (C₁₋₈)alkyl, optionally substituted by OH, (C₃₋₈)cycloalkyl, (C₁₋₃)alkyloxy, (C₁₋₃)alkylSO, (C₁₋₃)alkylSO₂, CN, NR₈R₉, COOR₁₀ or 1 or more halogens; or
R₂ is (C₃₋₁₀)cycloalkyl, (C₃₋₈)cycloalkenyl or (C₃₋₈)cycloalkyl(C₁₋₃)alkyl, each cycloalkyl group optionally substituted by (C₁₋₃)alkyl, hydroxy(C₁₋₃)alkyl, oxo, OH, ═NOH, COOR₁₀ or CN; or
R₂ is phenyl or pyridyl, each of which may be fused to a 5- or 6-membered saturated heterocyclic ring containing 1 or 2 heteroatoms selected from NR₁₁, O and S, and each of which may be substituted by NR₈R₉, halogen, OH, ═NOH, oxo, SH, (C₁₋₃)-alkyl, (C₁₋₃)alkyloxy or hydroxy(C₁₋₃)alkyl, each alkyl group optionally substituted by one or more halogens; or
R₂ together with R₇ and the N to which they are bonded form a saturated 4-8 membered ring, optionally containing a further heteroatom selected from O and S, the ring being optionally substituted by oxo, OH, COOR₁₀ or CONH₂; or
R₂ is (CH₂)ₙ-Het;
Het is a saturated 4-8-membered heterocyclic ring containing 1 or 2 heteroatoms selected from NR₁₁, O, S and SO₂, optionally substituted by OH or oxo;
n is 0, 1 or 2;
R₃ is (C₁₋₆)alkyl, (C₂₋₅)alkenyl, or (C₂₋₅)alkynyl, each of which optionally substituted by halogen, OH or phenyl; or
R₃ is (C₃₋₁₀)cycloalkyl, (C₃₋₈)cycloalkenyl or (C₃₋₈)cycloalkyl(C₁₋₃)alkyl, each cycloalkyl group may be fused to a benzo group, and each cycloalkyl group may be substituted by oxo, ═NOH, OH, COOR₁₂, CN, (C₁₋₃)alkyl or hydroxy(C₁₋₃)alkyl; or
R₃ is a saturated 4-8-membered heterocyclic ring containing 1 or 2 heteroatoms selected from N, O, S and SO₂, optionally substituted by hydroxyl or oxo; or
R₃ is phenyl or pyridyl, each of which may be fused to a 5- or 6-membered saturated heterocyclic ring containing 1 or 2 heteroatoms selected from NR₅, O and S, and each of which may be substituted by amino, halogen, hydroxy, hydroxyimino, oxo, mercapto, (C₁₋₃-alkyl, (C₁₋₃)-alkyloxy or hydroxy(C₁₋₃)alkyl, each alkyl group optionally substituted by one or more halogens; or
R₃ is a bicyclic heteroaromatic ring system containing 1-3 heteroatoms selected from N, O and S, which may be substituted by hydroxy, amino, (C₁₋₃)alkyl or hydroxy-(C₁₋₃)alkyl;
R₄ is H or (C₁₋₄)alkyl; or
R₄ together with R₃ and the N to which they are bonded form a saturated 4-8 membered ring, optionally containing a further heteroatom selected from O, S and SO₂, the ring being optionally substituted by oxo, hydroxyimino, hydroxy, carboxy, carboxamido, (C₁₋₃)alkyl, or hydroxy(C₁₋₃)alkyl or (C₁₋₄)-alkyloxy;
R₅, where present, is H or (C₁₋₄)alkyl;
R₆, where present, is H or (C₁₋₄)alkyl;
R₇ is H, (C₁₋₈)alkyl, (C₃₋₁₀)cycloalkyl, COR₁₃ or SO₂R₁₃;
R₈ and R₉ are independently H or (C₁₋₄)alkyl; or
R₈ and R₉ together with the N to which they are bonded form a 4-7-membered saturated heterocyclic ring;
R₁₀ is H or (C₁₋₄)alkyl;
R₁₁ is H or (C₁₋₄)alkyl;
R₁₂ is H or (C₁₋₄)alkyl;
R₁₃ is (C₁₋₆)alkyl;
or a pharmaceutically acceptable salt thereof.

12. The isoxazole-3-carboxamide derivative of claim 11, wherein R₁ is phenyl.

13. The isoxazole-3-carboxamide derivative of claim 12, wherein phenyl is substituted by 1-3 substituents selected from fluoro, chloro and CF₃.

14. The isoxazole-3-carboxamide derivative of claim 11, wherein X is NR₇.

15. The isoxazole-3-carboxamide derivative of claim 11, wherein R₂ is (C₁₋₈)alkyl, optionally substituted by OH, (C₃₋₆)cycloalkyl, (C₁₋₃)alkyloxy, (C₁₋₃)alkylSO, (C₁₋₃)alkylSO₂, CN, NR₈R₉, COOR₁₀ or 1 or more halogens; or
R₂ is (C₃₋₁₀)cycloalkyl, (C₃₋₈)cycloalkenyl or (C₃₋₈)cycloalkyl(C₁₋₃)alkyl, each cycloalkyl group optionally substituted by (C₁₋₃)alkyl, hydroxy(C₁₋₃)alkyl, oxo, OH, ═NOH, COOR₁₀ or CN.

16. The isoxazole-3-carboxamide derivative of claim 11, wherein R₃ is tetrahydropyranyl or (C₅₋₆)cycloalkyl, substituted by hydroxy or hydroxymethyl.

17. A pharmaceutical composition comprising an isoxazole-3-carboxamide derivative of claim 11 or a pharmaceutically acceptable salt thereof and pharmaceutically suitable auxiliaries.

18. A method of treating a human suffering from pain, wherein the pain is selected from the group consisting of acute and chronic pain disorders, acute and chronic neuropathic pain, acute and chronic inflammatory pain, respiratory diseases, and lower urinary tract disorders, the method comprising administering to the human a therapeutically effective amount of an isoxazole-3-carboxamide derivative of claim 11 or a pharmaceutically acceptable salt thereof.
